SEQUENCE LISTING (66 sequences)

SEQ ID NO: 1 - protein, 1397 aa, Artificial Sequence
  Synthetic
MEEISAAAVKVVPGPERPSPFSQLVYTSNDSYIVHSGDLRKIHKAASRGQVRKLEKMTKRKKTINLNIQDAQKRTALHWACVNGHEEVVTFLVDRKCQLDVLDGEHRTPLMKALQCHQEACANILIDSGADINLVDVYGNTALHYAVYSEILSVVAKLLSHGAVIEVHNKASLTPLLLSITKRSEQIVEFLLIKNANANAVNKYKCTALMLAVCHGSSEIVGMLLQQNVDVFAADICGVTAEHYAVTCGFHHIHEQIMEYIRKLSKNHQNTNPEGTSAGTPDEAAPLAERTPDTAESLVEKTPDEAAPLVERTPDTAESLVEKTPDEAASLVEGTSDKIQCLEKATSGKFEQSAEETPREITSPAKETSEKFTWPAKGRPRKIAWEKKEDTPREIMSPAKETSEKFTWAAKGRPRKIAWEKKETPVKTGCVARVTSNKTKVLEKGRSKMIACPTKESSTKASANDQRFPSESKQEEDEEYSCDSRSLFESSAKIQVCIPESIYQKVMEINREVEEPPKKPSAFKPAIEMQNSVPNKAFELKNEQTLRADPMFPPESKQKDYEENSWDSESLCETVSQKDVCLPKATHQKEIDKINGKLEESPNKDGLLKATCGMKVSIPTKALELKDMQTFKAEPPGKPSAFEPATEMQKSVPNKALELKNEQTLRADEILPSESKQKDYEENSWDTESLCETVSQKDVCLPKAAHQKEIDKINGKLEGSPVKDGLLKANCGMKVSIPTKALELMDMQTFKAEPPEKPSAFEPAIEMQKSVPNKALELKNEQTLRADEILPSESKQKDYEESSWDSESLCETVSQKDVCLPKATHQKEIDKINGKLEESPDNDGFLKAPCRMKVSIPTKALELMDMQTFKAEPPEKPSAFEPAIEMQKSVPNKALELKNEQTLRADQMFPSESKQKKVEENSWDSESLRETVSQKDVCVPKATHQKEMDKISGKLEDSTSLSKILDTVHSCERARELQKDHCEQRTGKMEQMKKKFCVLKKKLSEAKEIKSQLENQKVKWEQELCSVRLTLNQEEEKRRNADILNEKIREELGRIEEQHRKELEVKQQLEQALRIQDIELKSVESNLNQVSHTHENENYLLHENCMLKKEIAMLKLEIATLKHQYQEKENKYFEDIKILKEKNAELQMTLKLKEESLTKRASQYSGQLKVLIAENTMLTSKLKEKQDKEILEAEIESHHPRLASAVQDHDQIVTSRKSQEPAFHIAGDACLQRKMNVDVSSTIYNNEVLHQPLSEAQRKSKSLKINLNYAGDALRENTLVSEHAQRDQRETQCQMKEAEHMYQNEQDNVNKHTEQQESLDQKLFQLQSKNMWLQQQLVHAHKKADNKSKITIDIHFLERKMQHHLLKEKNEEIFNYNNHLKNRIYQYEKEKAETENS

SEQ ID NO: 2 - protein, 9 aa, Artificial Sequence
  Synthetic
SLSKILDTV

SEQ ID NO: 3 - protein, 23 aa, Artificial Sequence
  Synthetic
MVLLVTSLLLCELPHPAFLLIPD

SEQ ID NO: 4 - protein, 9 aa, Artificial Sequence
  Synthetic
GYSITSGYS

SEQ ID NO: 5 - protein, 7 aa, Artificial Sequence
  Synthetic
IHYSGDT

SEQ ID NO: 6 - protein, 8 aa, Artificial Sequence
  Synthetic
ATPGGFVY

SEQ ID NO: 7 - protein, 11 aa, Artificial Sequence
  Synthetic
QSLLDSDGKTY

SEQ ID NO: 8 - protein, 3 aa, Artificial Sequence
  Synthetic
LVS

SEQ ID NO: 9 - protein, 9 aa, Artificial Sequence
  Synthetic
WQGTHFPQT

SEQ ID NO: 10 - protein, 24 aa, Artificial Sequence
  Synthetic
VQLQESGPDLVKPSQSLSLTCTVT

SEQ ID NO: 11 - protein, 17 aa, Artificial Sequence
  Synthetic
WHWIRQFPGNKLEWMGY

SEQ ID NO: 12 - protein, 38 aa, Artificial Sequence
  Synthetic
DYNPSLKSRFSITRDTSKNQFFLQLNSVTTEDTATYYC

SEQ ID NO: 13 - protein, 11 aa, Artificial Sequence
  Synthetic
WGQGTLVTVSA

SEQ ID NO: 14 - protein, 26 aa, Artificial Sequence
  Synthetic
DVVMTQTPLTLSVTIGQPASISCKSS

SEQ ID NO: 15 - protein, 17 aa, Artificial Sequence
  Synthetic
LHWLLQRPGQSPKRLIF

SEQ ID NO: 16 - protein, 36 aa, Artificial Sequence
  Synthetic
KLDSGVPDRFTGSGSGTDFTLKISRVEAEDLGVYYC

SEQ ID NO: 17 - protein, 10 aa, Artificial Sequence
  Synthetic
FGGGTKLEIK

SEQ ID NO: 18 - protein, 114 aa, Artificial Sequence
  Synthetic
VQLQESGPDLVKPSQSLSLTCTVTGYSITSGYSWHWIRQFPGNKLEWMGYIHYSGDTDYNPSLKSRFSITRDTSKNQFFLQLNSVTTEDTATYYCATPGGFVYWGQGTLVTVSA

SEQ ID NO: 19 - protein, 112 aa, Artificial Sequence
  Synthetic
DVVMTQTPLTLSVTIGQPASISCKSSQSLLDSDGKTYLHWLLQRPGQSPKRLIFLVSKLDSGVPDRFTGSGSGTDFTLKISRVEAEDLGVYYCWQGTHFPQTFGGGTKLEIK

SEQ ID NO: 20 - protein, 15 aa, Artificial Sequence
  Synthetic
GGGGSGGGGSGGGGS

SEQ ID NO: 21 - protein, 241 aa, Artificial Sequence
  Synthetic
VQLQESGPDLVKPSQSLSLTCTVTGYSITSGYSWHWIRQFPGNKLEWMGYIHYSGDTDYNPSLKSRFSITRDTSKNQFFLQLNSVTTEDTATYYCATPGGFVYWGQGTLVTVSAGGGGSGGGGSGGGGSDVVMTQTPLTLSVTIGQPASISCKSSQSLLDSDGKTYLHWLLQRPGQSPKRLIFLVSKLDSGVPDRFTGSGSGTDFTLKISRVEAEDLGVYYCWQGTHFPQTFGGGTKLEIK

SEQ ID NO: 22 - protein, 66 aa, Artificial Sequence
  Synthetic
IEVMYPPPYLDNEKSNGTIIHVKGKHLCPSPLFPGPSKPFWVLVVVGGVLACYSLLVTVAFIIFWV

SEQ ID NO: 23 - protein, 41 aa, Artificial Sequence
  Synthetic
RSKRSRLLHSDYMNMTPRRPGPTRKHYQPYAPPRDFAAYRS

SEQ ID NO: 24 - protein, 107 aa, Artificial Sequence
  Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 25 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 463 aa, Artificial Sequence
  Synthetic
VQLQESGPDLVKPSQSLSLTCTVTGYSITSGYSWHWIRQFPGNKLEWMGYIHYSGDTDYNPSLKSRFSITRDTSKNQFFLQLNSVTTEDTATYYCATPGGFVYWGQGTLVTVSAGGGGSGGGGSGGGGSDVVMTQTPLTLSVTIGQPASISCKSSQSLLDSDGKTYLHWLLQRPGQSPKRLIFLVSKLDSGVPDRFTGSGSGTDFTLKISRVEAEDLGVYYCWQGTHFPQTFGGGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 83 aa, Artificial Sequence
  Synthetic
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 28 - protein, 47 aa, Artificial Sequence
  Synthetic
RFSVVKRGRKKLLYIFKQPFMRPVQTTQEEDGCSCRFPEEEEGGCEL

SEQ ID NO: 29 - protein, 527 aa, Artificial Sequence
  Synthetic
VQLQESGPDLVKPSQSLSLTCTVTGYSITSGYSWHWIRQFPGNKLEWMGYIHYSGDTDYNPSLKSRFSITRDTSKNQFFLQLNSVTTEDTATYYCATPGGFVYWGQGTLVTVSAGGGGSGGGGSGGGGSDVVMTQTPLTLSVTIGQPASISCKSSQSLLDSDGKTYLHWLLQRPGQSPKRLIFLVSKLDSGVPDRFTGSGSGTDFTLKISRVEAEDLGVYYCWQGTHFPQTFGGGTKLEIKAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - DNA, 69 bp, Artificial Sequence
  Synthetic
atggttctgctcgtgaccagcctgctgctgtgcgagctgcctcaccctgcctttctgctgatccccgac

SEQ ID NO: 31 - DNA, 27 bp, Artificial Sequence
  Synthetic
ggctacagcatcaccagcggctactct

SEQ ID NO: 32 - DNA, 21 bp, Artificial Sequence
  Synthetic
atccactacagcggcgacacc

SEQ ID NO: 33 - DNA, 24 bp, Artificial Sequence
  Synthetic
gccacccctggcggctttgtgtac

SEQ ID NO: 34 - DNA, 33 bp, Artificial Sequence
  Synthetic
cagtccctgctggacagcgacggcaagacctac

SEQ ID NO: 35 - DNA, 9 bp, Artificial Sequence
  Synthetic
ctggtgtct

SEQ ID NO: 36 - DNA, 27 bp, Artificial Sequence
  Synthetic
tggcagggcacccacttcccccagacc

SEQ ID NO: 37 - DNA, 72 bp, Artificial Sequence
  Synthetic
gtgcagctgcaggaaagcggccctgacctcgtgaagcctagccagagcctgtccctgacctgtaccgtgacc

SEQ ID NO: 38 - DNA, 51 bp, Artificial Sequence
  Synthetic
tggcactggatccggcagttccccggcaacaagctggaatggatgggctac

SEQ ID NO: 39 - DNA, 114 bp, Artificial Sequence
  Synthetic
gactacaaccccagcctgaagtcccggttctccatcacccgggacaccagcaagaaccagttttttctgcagctgaacagcgtgaccaccgaggacaccgccacctactactgt

SEQ ID NO: 40 - DNA, 33 bp, Artificial Sequence
  Synthetic
tggggccagggaacactcgtgaccgtgtctgct

SEQ ID NO: 41 - DNA, 78 bp, Artificial Sequence
  Synthetic
gatgtcgtgatgacccagacccccctgaccctgagcgtgacaattggccagcctgccagcatcagctgcaagagcagc

SEQ ID NO: 42 - DNA, 51 bp, Artificial Sequence
  Synthetic
ctgcattggctgctgcagaggccaggccagagccccaagagactgatcttc

SEQ ID NO: 43 - DNA, 108 bp, Artificial Sequence
  Synthetic
aagctggactccggcgtgcccgacagattcacaggcagcggctctggcaccgacttcaccctgaagatcagccgggtggaagccgaggacctgggcgtgtactactgt

SEQ ID NO: 44 - DNA, 30 bp, Artificial Sequence
  Synthetic
tttggaggcggcacaaagctggaaatcaaa

SEQ ID NO: 45 - DNA, 45 bp, Artificial Sequence
  Synthetic
ggcggcggaggatctggcggaggcggaagtggcggagggggatct

SEQ ID NO: 46 - DNA, 723 bp, Artificial Sequence
  Synthetic
gtgcagctgcaggaaagcggccctgacctcgtgaagcctagccagagcctgtccctgacctgtaccgtgaccggctacagcatcaccagcggctactcttggcactggatccggcagttccccggcaacaagctggaatggatgggctacatccactacagcggcgacaccgactacaaccccagcctgaagtcccggttctccatcacccgggacaccagcaagaaccagttttttctgcagctgaacagcgtgaccaccgaggacaccgccacctactactgtgccacccctggcggctttgtgtactggggccagggaacactcgtgaccgtgtctgctggcggcggaggatctggcggaggcggaagtggcggagggggatctgatgtcgtgatgacccagacccccctgaccctgagcgtgacaattggccagcctgccagcatcagctgcaagagcagccagtccctgctggacagcgacggcaagacctacctgcattggctgctgcagaggccaggccagagccccaagagactgatcttcctggtgtctaagctggactccggcgtgcccgacagattcacaggcagcggctctggcaccgacttcaccctgaagatcagccgggtggaagccgaggacctgggcgtgtactactgttggcagggcacccacttcccccagacctttggaggcggcacaaagctggaaatcaaa

SEQ ID NO: 47 - DNA, 321 bp, Artificial Sequence
  Synthetic
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 48 - DNA, 336 bp, Artificial Sequence
  Synthetic
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 49 - DNA, 1389 bp, Artificial Sequence
  Synthetic
gtgcagctgcaggaaagcggccctgacctcgtgaagcctagccagagcctgtccctgacctgtaccgtgaccggctacagcatcaccagcggctactcttggcactggatccggcagttccccggcaacaagctggaatggatgggctacatccactacagcggcgacaccgactacaaccccagcctgaagtcccggttctccatcacccgggacaccagcaagaaccagttttttctgcagctgaacagcgtgaccaccgaggacaccgccacctactactgtgccacccctggcggctttgtgtactggggccagggaacactcgtgaccgtgtctgctggcggcggaggatctggcggaggcggaagtggcggagggggatctgatgtcgtgatgacccagacccccctgaccctgagcgtgacaattggccagcctgccagcatcagctgcaagagcagccagtccctgctggacagcgacggcaagacctacctgcattggctgctgcagaggccaggccagagccccaagagactgatcttcctggtgtctaagctggactccggcgtgcccgacagattcacaggcagcggctctggcaccgacttcaccctgaagatcagccgggtggaagccgaggacctgggcgtgtactactgttggcagggcacccacttcccccagacctttggaggcggcacaaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 50 - DNA, 7008 bp, Artificial Sequence
  Synthetic
atggttctgctcgtgaccagcctgctgctgtgcgagctgcctcaccctgcctttctgctgatccccgacgtgcagctgcaggaaagcggccctgacctcgtgaagcctagccagagcctgtccctgacctgtaccgtgaccggctacagcatcaccagcggctactcttggcactggatccggcagttccccggcaacaagctggaatggatgggctacatccactacagcggcgacaccgactacaaccccagcctgaagtcccggttctccatcacccgggacaccagcaagaaccagttttttctgcagctgaacagcgtgaccaccgaggacaccgccacctactactgtgccacccctggcggctttgtgtactggggccagggaacactcgtgaccgtgtctgctggcggcggaggatctggcggaggcggaagtggcggagggggatctgatgtcgtgatgacccagacccccctgaccctgagcgtgacaattggccagcctgccagcatcagctgcaagagcagccagtccctgctggacagcgacggcaagacctacctgcattggctgctgcagaggccaggccagagccccaagagactgatcttcctggtgtctaagctggactccggcgtgcccgacagattcacaggcagcggctctggcaccgacttcaccctgaagatcagccgggtggaagccgaggacctgggcgtgtactactgttggcagggcacccacttcccccagacctttggaggcggcacaaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatccgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatccatcgatggagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgatttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgc

SEQ ID NO: 51 - DNA, 249 bp, Artificial Sequence
  Synthetic
ttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaac

SEQ ID NO: 52 - DNA, 123 bp, Artificial Sequence
  Synthetic
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 53 - DNA, 141 bp, Artificial Sequence
  Synthetic
cgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 54 - DNA, 1581 bp, Artificial Sequence
  Synthetic
gtgcagctgcaggaaagcggccctgacctcgtgaagcctagccagagcctgtccctgacctgtaccgtgaccggctacagcatcaccagcggctactcttggcactggatccggcagttccccggcaacaagctggaatggatgggctacatccactacagcggcgacaccgactacaaccccagcctgaagtcccggttctccatcacccgggacaccagcaagaaccagttttttctgcagctgaacagcgtgaccaccgaggacaccgccacctactactgtgccacccctggcggctttgtgtactggggccagggaacactcgtgaccgtgtctgctggcggcggaggatctggcggaggcggaagtggcggagggggatctgatgtcgtgatgacccagacccccctgaccctgagcgtgacaattggccagcctgccagcatcagctgcaagagcagccagtccctgctggacagcgacggcaagacctacctgcattggctgctgcagaggccaggccagagccccaagagactgatcttcctggtgtctaagctggactccggcgtgcccgacagattcacaggcagcggctctggcaccgacttcaccctgaagatcagccgggtggaagccgaggacctgggcgtgtactactgttggcagggcacccacttcccccagacctttggaggcggcacaaagctggaaatcaaagcggccgcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 55 - DNA, 7205 bp, Artificial Sequence
  Synthetic
atggttctgctcgtgaccagcctgctgctgtgcgagctgcctcaccctgcctttctgctgatccccgacgtgcagctgcaggaaagcggccctgacctcgtgaagcctagccagagcctgtccctgacctgtaccgtgaccggctacagcatcaccagcggctactcttggcactggatccggcagttccccggcaacaagctggaatggatgggctacatccactacagcggcgacaccgactacaaccccagcctgaagtcccggttctccatcacccgggacaccagcaagaaccagttttttctgcagctgaacagcgtgaccaccgaggacaccgccacctactactgtgccacccctggcggctttgtgtactggggccagggaacactcgtgaccgtgtctgctggcggcggaggatctggcggaggcggaagtggcggagggggatctgatgtcgtgatgacccagacccccctgaccctgagcgtgacaattggccagcctgccagcatcagctgcaagagcagccagtccctgctggacagcgacggcaagacctacctgcattggctgctgcagaggccaggccagagccccaagagactgatcttcctggtgtctaagctggactccggcgtgcccgacagattcacaggcagcggctctggcaccgacttcaccctgaagatcagccgggtggaagccgaggacctgggcgtgtactactgttggcagggcacccacttcccccagacctttggaggcggcacaaagctggaaatcaaagcggccgcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatccgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatccatcgatggagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgatttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgctcgag

SEQ ID NO: 56 - protein, 236 aa, Artificial Sequence
  Synthetic
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 57 - DNA, 708 bp, Artificial Sequence
  Synthetic
gagcctaagtcgtgcgataagacgcatacttgtcccccgtgtcctgctccagaactacttggtgggccatctgtgttcctcttcccgcccaagccaaaggataccctcatgatttcccgcacaccagaggtcacgtgcgtagtcgttgatgtgtcccatgaggatcctgaagtgaagtttaattggtacgttgacggtgtagaggtacacaacgctaagacaaagccccgcgaagagcaatacaactcgacgtatagggttgtcagcgtactcaccgttcttcatcaggactggcttaatggtaaagaatacaagtgtaaagtttcaaacaaagctctcccggctccgatagaaaaaacaataagcaaagcgaaaggccagcctcgggaacctcaggtatacactctcccaccttcgcgtgacgagttaacaaagaatcaggtatccctgacctgtctggtcaagggtttctacccctccgacatcgcggtagaatgggaaagtaatggacagcctgagaacaactataaaactactccccctgtcttggatagtgatggctcattcttcctatactctaagttgactgtcgataagtcgaggtggcagcagggaaatgtgttctcatgcagcgtaatgcatgaggctcttcacaaccattacacccaaaagagccttagccttagtccggggaagaaggatcccaaa

SEQ ID NO: 58 - DNA, 792 bp, Artificial Sequence
  Synthetic
atggttctgctggtcaccagcctgctgctgtgcgaactgccccaccccgcctttctgctgatccccgatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctacatacactacagcggtgacactgactacaacccatctctcaaaagtcgattctctatcactcgcgacacatccaagaaccagttcttcctgcagttgaattctgtgactactgaggacacagccacatattactgtgcaaccccgggggggtttgtttactggggccaagggactctggtcactgtctctgcaggcggcggagggagtggaggcggaggatctggcggcgggggatccgatgttgtgatgacccagactccactcactttgtcggttaccattggacaaccagcctccatctcttgcaagtcaagtcagagcctcttagatagtgatggaaagacatatttgcattggttgttacagaggccaggccagtctccaaagcgcctaatctttctggtgtctaaactggactctggagtccctgacaggttcactggcagtggatcagggacagatttcacactgaaaatcagcagagtggaggctgaggatttgggagtgtattattgctggcaaggtacacattttcctcagacgttcggtggaggcaccaagctggaaatcaaa

SEQ ID NO: 59 - protein, 23 aa, Artificial Sequence
  Synthetic
MLLLVTSLLLCELPHPAFLLIPD

SEQ ID NO: 60 - protein, 486 aa, Artificial Sequence
  Synthetic
VQLQESGPDLVKPSQSLSLTCTVTGYSITSGYSWHWIRQFPGNKLEWMGYIHYSGDTDYNPSLKSRFSITRDTSKNQFFLQLNSVTTEDTATYYCATPGGFVYWGQGTLVTVSAGGGGSGGGGSGGGGSDVVMTQTPLTLSVTIGQPASISCKSSQSLLDSDGKTYLHWLLQRPGQSPKRLIFLVSKLDSGVPDRFTGSGSGTDFTLKISRVEAEDLGVYYCWQGTHFPQTFGGGTKLEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - DNA, 1527 bp, Artificial Sequence
  Synthetic
atgcttctgctggtcaccagcctgctgctgtgcgaactgccccaccccgcctttctgctgatccccgatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctacatacactacagcggtgacactgactacaacccatctctcaaaagtcgattctctatcactcgcgacacatccaagaaccagttcttcctgcagttgaattctgtgactactgaggacacagccacatattactgtgcaaccccgggggggtttgtttactggggccaagggactctggtcactgtctctgcaggcggcggagggagtggaggcggaggatctggcggcgggggatccgatgttgtgatgacccagactccactcactttgtcggttaccattggacaaccagcctccatctcttgcaagtcaagtcagagcctcttagatagtgatggaaagacatatttgcattggttgttacagaggccaggccagtctccaaagcgcctaatctttctggtgtctaaactggactctggagtccctgacaggttcactggcagtggatcagggacagatttcacactgaaaatcagcagagtggaggctgaggatttgggagtgtattattgctggcaaggtacacattttcctcagacgttcggtggaggcaccaagctggaaatcaaagcggccgcattcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaaccgcttcagcgtcgtgaagcggggcagaaagaagctgctgtacatcttcaagcagcccttcatgcggcccgtgcagaccacccaggaagaggacggctgctcctgcagattccccgaggaagaagaaggcggctgcgagctgagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 62 - DNA, 792 bp, Artificial Sequence
  Synthetic
atgcttctgctggtcaccagcctgctgctgtgcgaactgccccaccccgcctttctgctgatccccgatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctacatacactacagcggtgacactgactacaacccatctctcaaaagtcgattctctatcactcgcgacacatccaagaaccagttcttcctgcagttgaattctgtgactactgaggacacagccacatattactgtgcaaccccgggggggtttgtttactggggccaagggactctggtcactgtctctgcaggcggcggagggagtggaggcggaggatctggcggcgggggatccgatgttgtgatgacccagactccactcactttgtcggttaccattggacaaccagcctccatctcttgcaagtcaagtcagagcctcttagatagtgatggaaagacatatttgcattggttgttacagaggccaggccagtctccaaagcgcctaatctttctggtgtctaaactggactctggagtccctgacaggttcactggcagtggatcagggacagatttcacactgaaaatcagcagagtggaggctgaggatttgggagtgtattattgctggcaaggtacacattttcctcagacgttcggtggaggcaccaagctggaaatcaaa

SEQ ID NO: 63 - DNA, 249 bp, Artificial Sequence
  Synthetic
ttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaac

SEQ ID NO: 64 - DNA, 141 bp, Artificial Sequence
  Synthetic
cgcttcagcgtcgtgaagcggggcagaaagaagctgctgtacatcttcaagcagcccttcatgcggcccgtgcagaccacccaggaagaggacggctgctcctgcagattccccgaggaagaagaaggcggctgcgagctg

SEQ ID NO: 65 - DNA, 336 bp, Artificial Sequence
  Synthetic
agagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 66 - DNA, 7086 bp, Artificial Sequence
  Synthetic
ccctcgaggccgccatgcttctgctggtcaccagcctgctgctgtgcgaactgccccaccccgcctttctgctgatccccgatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctacatacactacagcggtgacactgactacaacccatctctcaaaagtcgattctctatcactcgcgacacatccaagaaccagttcttcctgcagttgaattctgtgactactgaggacacagccacatattactgtgcaaccccgggggggtttgtttactggggccaagggactctggtcactgtctctgcaggcggcggagggagtggaggcggaggatctggcggcgggggatccgatgttgtgatgacccagactccactcactttgtcggttaccattggacaaccagcctccatctcttgcaagtcaagtcagagcctcttagatagtgatggaaagacatatttgcattggttgttacagaggccaggccagtctccaaagcgcctaatctttctggtgtctaaactggactctggagtccctgacaggttcactggcagtggatcagggacagatttcacactgaaaatcagcagagtggaggctgaggatttgggagtgtattattgctggcaaggtacacattttcctcagacgttcggtggaggcaccaagctggaaatcaaagcggccgcattcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaaccgcttcagcgtcgtgaagcggggcagaaagaagctgctgtacatcttcaagcagcccttcatgcggcccgtgcagaccacccaggaagaggacggctgctcctgcagattccccgaggaagaagaaggcggctgcgagctgagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatccgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatccatcgatggagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgatttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctag

(19) United States
(12) Patent Application Publication
(10) Pub. No.: US 2016/0333422 A1
(43) Pub. Date: Nov. 17, 2016

(54) ANTI-NY-BR-1 POLYPEPTIDES, PROTEINS, AND CHIMERIC ANTIGEN RECEPTORS

(71) Applicant: THE UNITED STATES OF AMERICA, AS REPRESENTED BY THE SECRETARY, DEPARTMENT OF HEALTH AND HUMAN SERV, Bethesda, MD (US)

(72) Inventors: Steven A. Feldman, Washington, DC (US); Steven A. Rosenberg, Potomac, MD (US)

(21) Appl. No.: 15/112,524

(22) PCT Filed: Jan. 23, 2015
(86) PCT No.: PCT/US2015/012633

§ 371 Date: Jul. 19, 2016

Related U.S. Application Data
(60) Provisional application No. 61/931,095, filed on Jan. 24, 2014.

(51) Int. Cl. C12Q 1/68; G01N 33/574; C07K 14/725; C07K 16/30; C07K 14/705

(52) U.S. Cl. CPC C12Q 1/6886; C07K 16/3015; C07K 14/70521; C07K 14/7051; G01N 33/57484; C07K 2319/00; C07K 2319/03; C07K 2319/02; C07K 2317/622; A61K 38/00

(57) ABSTRACT

Polypeptides and proteins that specifically bind to and immunologically recognize NY-BR-1 are disclosed. Chimeric antigen receptors (CARs), anti-NY-BR-1 binding moieties, nucleic acids, recombinant expression vectors, host cells, populations of cells, and pharmaceutical compositions relating to the polypeptides and proteins are also disclosed. Methods of detecting the presence of cancer in a mammal and methods of treating or preventing cancer in a mammal are also disclosed.

### CROSS-REFERENCE TO RELATED APPLICATION

This patent application claims the benefit of U.S. Provisional Patent Application No. 61/931,095, filed Jan. 24, 2014, which is incorporated by reference in its entirety herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 86,829 Byte ASCII (Text) file named “719505ST25.TXT,” dated Jan. 5, 2015.

### BACKGROUND OF THE INVENTION

Cancer is a public health concern. Despite advances in treatments such as chemotherapy, the prognosis for many cancers, including breast cancer, may be poor. It is estimated that about 559,650 Americans will die from cancer, corresponding to 1,500 deaths per day (Jemal et al., CA Cancer J. Clin., 57:43-66 (2007)). Accordingly, there exists an unmet need for additional treatments for cancer, particularly breast cancer.

### BRIEF SUMMARY OF THE INVENTION

An embodiment of the invention provides an isolated polypeptide comprising the amino acid sequences of SEQ ID NOs: 4-9.

Another embodiment of the invention provides an isolated protein comprising a first polypeptide chain comprising the amino acid sequences of SEQ ID NOs: 4-6 and a second polypeptide chain comprising the amino acid sequences of SEQ ID NOs: 7-9.

Still another embodiment of the invention provides a chimeric antigen receptor (CAR) comprising an antigen binding domain having antigenic specificity for NY-BR-1, a transmembrane domain, and an intracellular T cell signaling domain.

Further embodiments of the invention provide related anti-NY-BR-1 binding moieties, nucleic acids, recombinant expression vectors, host cells, populations of cells, conjugates, and pharmaceutical compositions relating to the polypeptides, proteins, and CARs of the invention.

Additional embodiments of the invention provide methods of detecting the presence of cancer in a mammal and methods of treating or preventing cancer in a mammal.

### BRIEF DESCRIPTION OF THE SEVERAL VIEWS OF THE DRAWINGS

FIGS. 1A and 1B are graphs showing the number of copies of NY-BR-1 (ANKRD30A) mRNA per 10⁵ copies of beta-actin mRNA in normal tissues of the human body (A) and normal human brain tissue (B).

FIG. 2 is a graph showing the number of copies of NY-BR-1 (ANKRD30A) mRNA per 10⁵ copies of beta-actin mRNA in tumor cell lines.

FIG. 3 is a graph showing the number of copies of NY-BR-1 (ANKRD30A) mRNA per 10⁵ copies of beta-actin mRNA in various tissues of a breast cancer tissue quantitative polymerase chain reaction (qPCR) panel.

FIG. 4 is a graph showing the number of copies of NY-BR-1 (ANKRD30A) mRNA per 10⁵ copies of beta-actin mRNA molecules in target cell lines that were untransfected or transfected with an expression vector encoding NY-BR-1.

### DETAILED DESCRIPTION OF THE INVENTION

An embodiment of the invention provides polypeptides and proteins comprising an antigen binding domain of an anti-NY-BR-1antibody. The polypeptides and proteins advantageously specifically recognize and bind to NY-BR-1 (also known as ANKRD30A). NY-BR-1 is expressed on the cell surface. In normal tissues, NY-BR-1 expression is detected in normal breast, placenta and testis. NY-BR-1 is overexpressed in breast cancer, including primary and metastatic breast cancer tumors.

Without being bound to a particular theory or mechanism, it is believed that by specifically recognizing and binding to NY-BR-1, the inventive polypeptides and proteins may, advantageously, target NY-BR-1-expressing cancer cells. In an embodiment of the invention, the inventive polypeptides and proteins may elicit an antigen-specific response against NY-BR-1. Accordingly, without being bound to a particular theory or mechanism, it is believed that by eliciting an antigen-specific response against NY-BR-1, the inventive proteins and polypeptides may provide for one or more of the following: targeting and destroying NY-BR-1-expressing cancer cells, reducing or eliminating cancer cells, facilitating infiltration of immune cells and/or effector molecules to tumor site(s), and enhancing/extending anti-cancer responses. Without being bound to a particular theory or mechanism, the inventive polypeptides and proteins may, advantageously, mediate immune reactions through antibody-dependent cellular cytoxicity (ADCC).

In an embodiment, the inventive polypeptides and proteins specifically recognize and bind to the NY-BR-1 amino acid sequence of SEQ ID NO: 1. Preferably, the inventive polypeptides and proteins specifically recognize and bind to the NY-BR-1 amino acid sequence of SEQ ID NO: 2 (amino acid residues 960-968 of NY-BR-1 SEQ ID NO: 1).

The term “polypeptide,” as used herein, includes oligopeptides and refers to a single chain of amino acids connected by one or more peptide bonds. The polypeptide may comprise one or more variable regions (e.g., two variable regions) of an antigen binding domain of an anti-NY-BR-1 antibody, each variable region comprising a complementarity determining region (CDR) 1, a CDR2, and a CDR3. In an embodiment of the invention, the first variable region may be a heavy chain and the second variable region may be a light chain. Preferably, a first variable region comprises a CDR1 comprising the amino acid sequence of SEQ ID NO: 4 (CDR1 of first variable region), a CDR2 comprising the amino acid sequence of SEQ ID NO: 5 (CDR2 of first variable region), and a CDR3 comprising the amino acid sequence of SEQ ID NO: 6 (CDR3 of first variable region), and the second variable region comprises a CDR1 comprising the amino acid sequence of SEQ ID NO: 7 (CDR1 of second variable region), a CDR2 comprising the amino acid sequence of SEQ ID NO: 8 (CDR2 of second variable region), and a CDR3 comprising the amino acid sequence of SEQ ID NO: 9 (CDR3 of second variable region). In this regard, the inventive polypeptide can comprise the amino acid sequences of SEQ ID NOs: 4-6, 7-9, or 4-9. Preferably, the polypeptide comprises the amino acid sequences of SEQ ID NOs: 4-9.

In an embodiment, each variable region of the antigen binding domain comprises a framework region (FR) 1, an FR2, an FR3, and an FR4 in addition to the CDR regions described above. Preferably, the first variable region comprises an FR1 comprising the amino acid sequence of SEQ ID NO: 10 (FR1 of first variable region), an FR2 comprising the amino acid sequence of SEQ ID NO: 11 (FR2 of first variable region), an FR3 comprising the amino acid sequence of SEQ ID NO: 12 (FR3 of first variable region), and an FR4 comprising the amino acid sequence of SEQ ID NO: 13 (FR4 of first variable region), and the second variable region comprises an FR1 comprising the amino acid sequence of SEQ ID NO: 14 (FR1 of second variable region), an FR2 comprising the amino acid sequence of SEQ ID NO: 15 (FR2 of second variable region), an FR3 comprising the amino acid sequence of SEQ ID NO: 16 (FR3 of second variable region), and an FR4 comprising the amino acid sequence of SEQ ID NO: 17 (FR4 of second variable region). In this regard, the inventive polypeptide can comprise the amino acid sequences of SEQ ID NOs: 10-13, SEQ ID NOs: 14-17, SEQ ID NOs: 10-17, SEQ ID NOs: 4-6 and 10-13, SEQ ID NOs: 7-9 and 14-17, or SEQ ID NOs: 4-17. Preferably, the polypeptide comprises the amino acid sequences of SEQ ID NOs: 4-17.

In an embodiment, the polypeptides each comprise one or more variable regions (e.g., first and second variable regions) of an antigen binding domain of an anti-NY-BR-1 antibody, each comprising the CDRs and FRs as described above. The first variable region may comprise the amino acid sequence of SEQ ID NO: 18. The second variable region may comprise the amino acid sequence of SEQ ID NO: 19. Accordingly, in an embodiment of the invention, the polypeptide comprises the amino acid sequence(s) of SEQ ID NO: 18, SEQ ID NO: 19, or SEQ ID NOs: 18 and 19. Preferably, the polypeptide comprises the amino acid sequences of SEQ ID NOs: 18 and 19.

In an embodiment of the invention, the first and second variable regions of the polypeptide may be joined by a linker amino acid sequence. The linker amino acid sequence may comprise any suitable amino acid sequence. In an embodiment of the invention, the linker amino acid sequence is not naturally occurring as a whole. In an embodiment of the invention, the linker amino acid sequence may comprise the amino acid sequence of SEQ ID NO: 20.

In an embodiment of the invention, the polypeptide comprises first and second variable regions, each comprising the CDRs and FRs as described above, with a linker amino acid sequence positioned between the first and second variable regions. In an embodiment, the light chain variable region is positioned on the amino terminus of the polypeptide and the heavy chain variable region is positioned on the carboxyl terminus of the polypeptide, with the linker amino acid sequence positioned between them. Preferably, however, the heavy chain variable region is positioned on the amino terminus and the light chain variable region is positioned on the carboxyl terminus, with the linker amino acid sequence positioned between them. In this regard, the polypeptide may comprise the amino acid sequence of SEQ ID NO: 21.

In an embodiment, the polypeptide comprises a leader amino acid sequence. The leader sequence may be positioned at the amino terminus of the polypeptide. The leader amino acid sequence may comprise any suitable leader amino acid sequence. Examples of leader amino acid sequences include, but are not limited to, human IgG kappa light chain leader sequence, human CD8 leader sequence, and human granulocyte-macrophage colony-stimulating factor (GM-CSF) receptor leader sequence. In an embodiment, the leader sequence is a human GM-CSF receptor sequence. The leader amino acid sequence may comprise, for example, the amino acid sequence of SEQ ID NO: 3 or 59. In an embodiment of the invention, while the leader sequence may facilitate expression of the polypeptide on the surface of the cell, the presence of the leader sequence in an expressed polypeptide is not necessary in order for the polypeptide to function. In an embodiment of the invention, upon expression of the polypeptide on the cell surface, the leader sequence may be cleaved off of the polypeptide. Accordingly, in an embodiment of the invention, the polypeptide lacks a leader sequence.

The invention further provides a protein comprising at least one of the polypeptides described herein. By “protein” is meant a molecule comprising one or more polypeptide chains.

The protein of the invention can comprise a first polypeptide chain comprising the amino acid sequences of SEQ ID NOs: 4-6 and a second polypeptide chain comprising the amino acid sequences of SEQ ID NOs: 7-9. In an embodiment of the invention, the first polypeptide chain further comprises the amino acid sequences of SEQ ID NOs: 10-13 and the second polypeptide chain further comprises the amino acid sequences of SEQ ID NOs: 14-17. In this regard, the protein of the invention can comprise a first polypeptide chain comprising the amino acid sequences of SEQ ID NOs: 4-6 and 10-13 and a second polypeptide chain comprising the amino acid sequences of SEQ ID NOs: 7-9 and 14-17. In an embodiment of the invention, the protein of the invention comprises a first polypeptide chain comprising the amino acid sequence of SEQ ID NO: 18 and the second polypeptide chain comprises the amino acid sequence of SEQ ID NO: 19. In still another embodiment of the invention, the protein comprises the amino acid sequence of SEQ ID NO: 21.

The protein may further comprise a leader sequence and/or a linker sequence as described herein with respect to other aspects of the invention. In an embodiment, the protein lacks a leader sequence.

The protein of the invention can be, for example, a fusion protein. If, for example, the protein comprises a single polypeptide chain comprising (i) SEQ ID NO: 18 and (ii) SEQ ID NO: 19, or if the first and/or second polypeptide chain(s) of the protein further comprise(s) other amino acid sequences, e.g., an amino acid sequence encoding an immunoglobulin or a portion thereof, then the inventive protein can be a fusion protein. In this regard, the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin, CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods. See, for instance, Choi et al., Mol. Biotechnol. 31: 193-202 (2005).

It is contemplated that the polypeptides and proteins of the invention may be useful as anti-NY-BR-1 binding moieties. In this regard, an embodiment of the invention provides an anti-NY-BR-1 binding moiety comprising any of the polypeptides or proteins described herein. In an embodiment of the invention, the anti-NY-BR-1 binding moiety comprises an antigen binding portion of any of the polypeptides or proteins described herein. The antigen binding portion can be any portion that has at least one antigen binding site. In an embodiment, the anti-NY-BR-1 binding moiety is a Fab fragment (Fab), F(ab′)₂ fragment, diabody, triabody, tetrabody, single-chain variable region fragment (scFv), or disulfide-stabilized variable region fragment (dsFv).

In an embodiment, the anti-NY-BR-1 binding moiety can be an antibody. The antibody may be, for example, a recombinant antibody comprising at least one of the inventive polypeptides described herein. As used herein, “recombinant antibody” refers to a recombinant (e.g., genetically engineered) protein comprising at least one of the polypeptides or proteins of the invention and one or more polypeptide chains of an antibody, or a portion thereof. The polypeptide of an antibody, or portion thereof, can be, for example, a constant region of a heavy or light chain, or an Fc fragment of an antibody, etc. The polypeptide chain of an antibody, or portion thereof, can exist as a separate polypeptide of the recombinant antibody. Alternatively, the polypeptide chain of an antibody, or portion thereof, can exist as a polypeptide, which is expressed in frame (in tandem) with the polypeptide or protein of the invention. The polypeptide of an antibody, or portion thereof, can be a polypeptide of any antibody or any antibody fragment.

The antibody of the invention can be any type of immunoglobulin that is known in the art. For instance, the anti-NY-BR-1 binding moiety can be an antibody of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for NY-BR-1.

Methods of testing antibodies for the ability to bind to NY-BR-1 are known in the art and include any antibody-antigen binding assay, such as, for example, radioimmunoassay (RIA), ELISA, Western blot, immunoprecipitation, and competitive inhibition assays (see, e.g., Murphy et al., infra).

Suitable methods of making antibodies are known in the art. For instance, standard hybridoma methods are described in, e.g., Köhler and Milstein, Eur. J. Immunol., 5, 511-519 (1976), Greenfield (ed.), Antibodies: A Laboratory Manual, CSH Press (2013), and Murphy et al. (eds.), Janeway's Immunobiology, 8^{th} Ed., Garland Science, New York, N.Y. (2011). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352.

Phage display furthermore can be used to generate an antibody. In this regard, phage libraries encoding antigen-binding variable (V) domains of antibodies can be generated using standard molecular biology and recombinant DNA techniques. See, for instance, Green et al. (eds.), Molecular Cloning, A Laboratory Manual, 4^{th} Edition, Cold Spring Harbor Laboratory Press, New York (2012) and Ausubel et al., Current Protocols in Molecular Biology, Greene Publishing Associates and John Wiley & Sons, NY (2007). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Murphy et al., supra, Huse et al., supra, and U.S. Pat. No. 6,265,150).

Antibodies can be produced by transgenic mice that are transgenic for specific heavy and light chain immunoglobulin genes. Such methods are known in the art and described in, for example U.S. Pat. Nos. 5,545,806 and 5,569,825, and Murphy et al., supra.

Methods for generating humanized antibodies are well known in the art and are described in detail in, for example, Murphy et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235, 959-973 (1994).

In a preferred embodiment, the anti-NY-BR-1 binding moiety is a single-chain variable region fragment (scFv). A single-chain variable region fragment (scFv) antibody fragment, which is a truncated Fab fragment including the variable (V) domain of an antibody heavy chain linked to a V domain of a light antibody chain via a synthetic peptide, can be generated using routine recombinant DNA technology techniques (see, e.g., Murphy et al., supra). Similarly, disulfide-stabilized variable region fragments (dsFv) can be prepared by recombinant DNA technology (see, e.g., Reiter et al., Protein Engineering, 7, 697-704 (1994)). The anti-NY-BR-1 binding moieties of the invention, however, are not limited to these exemplary types of antibody fragments.

Also, the anti-NY-BR-1 binding moiety can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

Another embodiment of the invention provides chimeric antigen receptors (CARs) comprising: (a) an antigen binding domain having antigenic specificity for NY-BR-1, (b) a transmembrane domain, and (c) an intracellular T cell signaling domain.

A chimeric antigen receptor (CAR) is an artificially constructed hybrid protein or polypeptide containing the antigen binding domains of an antibody (e.g., single chain variable fragment (scFv)) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives cells expressing CARs the ability to recognize antigen independent of antigen processing, thus bypassing a major mechanism of tumor escape. Because the CARs are not MHC-restricted, they may be used to treat patients expressing any MHC allele, thereby enlarging the patient population that may be treated. Moreover, when expressed in T-cells, CARs advantageously do not dimerize with endogenous T cell receptor (TCR) alpha and beta chains.

The phrases “have antigenic specificity” and “elicit antigen-specific response,” as used herein, means that the CAR can specifically bind to and immunologically recognize an antigen, such that binding of the CAR to the antigen elicits an immune response.

The CARs of the invention have antigenic specificity for NY-BR-1. Without being bound to a particular theory or mechanism, it is believed that by eliciting an antigen-specific response against NY-BR-1, the inventive CARs provide for one or more of the following: targeting and destroying NY-BR-1-expressing cancer cells, reducing or eliminating cancer cells, facilitating infiltration of immune cells to tumor site(s), and enhancing/extending anti-cancer responses.

An embodiment of the invention provides a CAR comprising an antigen binding domain of an anti-NY-BR-1 antibody. The antigen binding domain of the anti-NY-BR-1 antibody specifically recognizes and binds to NY-BR-1 as described herein with respect to other aspects of the invention. The antigen binding domain of the CARs may comprise any of the polypeptides or proteins described herein with respect to other aspects of the invention. In an embodiment of the invention, the CAR comprises an anti-NY-BR-1 single chain variable fragment (scFv). In this regard, a preferred embodiment of the invention provides CARs comprising an antigen-binding domain comprising a single chain variable fragment (scFv) that comprises any of the polypeptides or proteins described herein.

In an embodiment, the CAR comprises an immunoglobulin constant domain.

Preferably, the immunoglobulin domain is a human immunoglobulin sequence. In an embodiment, the immunoglobulin constant domain comprises an immunoglobulin CH2 and CH3 immunoglobulin G (IgG1) domain sequence (CH2CH3). In this regard, the CAR may comprise an immunoglobulin constant domain (CH2CH3) comprising SEQ ID NO: 56. Without being bound to a particular theory or mechanism, it is believed that the CH2CH3 domain may extend the binding motif of the scFv away from the membrane of the CAR-expressing cells, may more accurately mimic the size and domain structure of a native TCR, and may provide greater flexibility for the scFv to bind to NY-BR-1. In some embodiments, the CAR may lack an immunoglobulin constant domain.

In an embodiment of the invention, the CAR comprises a transmembrane domain. In an embodiment of the invention, the transmembrane domain comprises (i) a CD28 transmembrane amino acid sequence, (ii) a CD8 transmembrane amino acid sequence, or both (i) and (ii). In a preferred embodiment, the CD8 and CD28 transmembrane amino acid sequences are human sequences. The CD8 or CD28 transmembrane amino acid sequence may comprise less than the whole CD8 or CD28 amino acid sequence, respectively, and may include a “hinge” sequence. In this regard, the CD28 transmembrane amino acid sequence may comprise the amino acid sequence of SEQ ID NO: 22 and the CD8 transmembrane amino acid sequence may comprise the amino acid sequence of SEQ ID NO: 27.

In an embodiment of the invention, the CAR comprises an intracellular T cell signaling domain comprising a CD28 intracellular T cell signaling amino acid sequence, a CD3ζ intracellular T cell signaling amino acid sequence, a 4-1BB intracellular T cell signaling amino acid sequence, or a combination of two or more of the foregoing.

In a preferred embodiment, the CD28 intracellular T cell signaling amino acid sequence, the CD3ζ intracellular T cell signaling amino acid sequence, and the 4-1BB intracellular T cell signaling amino acid sequence are human sequences. CD28 is a T cell marker important in T cell co-stimulation. 4-1BB, also known as CD137, transmits a potent costimulatory signal to T cells, promoting differentiation and enhancing long-term survival of T lymphocytes. CD3ζ associates with TCRs to produce a signal and contains immunoreceptor tyrosine-based activation motifs (ITAMs). The CD28 intracellular T cell signaling amino acid sequence, the CD3ζ intracellular T cell signaling amino acid sequence, and the 4-1BB intracellular T cell signaling amino acid sequence may comprise less than the whole CD28, 4-1BB, or CD3ζ respectively. In an embodiment of the invention, the CD28 intracellular T cell signaling sequence comprises the amino acid sequence of SEQ ID NO: 23, the CD3ζ intracellular T cell signaling sequence comprises the amino acid sequence of SEQ ID NO: 25, and the 4-1BB intracellular T cell signaling sequence comprises the amino acid sequence of SEQ ID NO: 28.

In an embodiment of the invention, the CAR comprises both a CD28 transmembrane sequence and a CD28 intracellular T cell signaling sequence. In this regard, the CAR may comprise a CD28 transmembrane sequence and intracellular T cell signaling sequence comprising the amino acid sequence of SEQ ID NO: 24.

In an embodiment of the invention, the CAR comprises a CD28 transmembrane sequence, a CD28 intracellular T cell signaling sequence, and a CD3ζ intracellular T cell signaling sequence (also referred to as a “second generation” CAR herein). In this regard, the second generation CAR may comprise a CD28 transmembrane amino acid sequence comprising the amino acid sequence of SEQ ID NO: 22, a CD28 intracellular T cell signaling amino acid sequence comprising the amino acid sequence of SEQ ID NO: 23, and a CD3ζ intracellular T cell signaling amino acid sequence comprising the amino acid sequence of SEQ ID NO: 25. In another embodiment of the invention, the second generation CAR may comprise a CD28 transmembrane and intracellular T cell signaling amino acid sequence comprising the amino acid sequence of SEQ ID NO: 24 and a CD3ζ intracellular T cell signaling amino acid sequence comprising the amino acid sequence of SEQ ID NO: 25. In another embodiment of the invention, the second generation CAR may comprise the amino acid sequence(s) of (a) SEQ ID NOs: 4-9, 22, 23, and 25; (b) SEQ ID NOs: 4-9, 24, and 25; (c) SEQ ID NOs: 3-9, 22, 23, and 25; (d) SEQ ID NOs: 3-9, 24, and 25; (e) SEQ ID NOs: 4-17, 22, 23, and 25; (f) SEQ ID NOs: 4-17, 24, and 25; (g) SEQ ID NOs: 3-17, 22, 23, and 25; (h) SEQ ID NOs: 3-17, 24, and 25; (i) SEQ ID NOs: 18, 19, 22, 23, and 25; (j) SEQ ID NOs: 18, 19, 24, and 25; (k) SEQ ID NOs: 3, 18, 19, 22, 23, and 25; (1) SEQ ID NOs: 3, 18, 19, 24, and 25; (m) SEQ ID NOs: 21, 22, 23, and 25; (n) SEQ ID NOs: 21, 24, and 25; (o) SEQ ID NOs: 3, 21, 22, 23, and 25; (p) SEQ ID NOs: 3, 21, 24, and 25; (q) SEQ ID NO: 26; or (r) SEQ ID NOs: 3 and 26. Preferably, the second generation CAR comprises the amino acid sequence of SEQ ID NO: 26.

In an embodiment of the invention, the CAR comprises a CD8 transmembrane sequence, a CD28 intracellular T cell signaling sequence, a 4-1BB intracellular T cell signaling sequence, and a CD3ζ intracellular T cell signaling sequence (also referred to as a “third generation” CAR herein). In this regard, the CAR may comprise a CD8 transmembrane amino acid sequence comprising the amino acid sequence of SEQ ID NO: 27, a CD28 intracellular T cell signaling amino acid sequence comprising the amino acid sequence of SEQ ID NO: 23, a 4-1BB intracellular T cell signaling amino acid sequence comprising the amino acid sequence of SEQ ID NO: 28, and a CD3ζ intracellular T cell signaling amino acid sequence comprising the amino acid sequence of SEQ ID NO: 25. In another embodiment of the invention, the second generation CAR may comprise the amino acid sequence(s) of (a) SEQ ID NOs: 4-9, 23, 25, 27, and 28; (b) SEQ ID NOs: 3-9, 23, 25, 27, and 28; (c) SEQ ID NOs: 4-17, 23, 25, 27, and 28; (d) SEQ ID NOs: 3-17, 23, 25, 27, and 28; (e) 18, 19, 23, 25, 27, and 28; (f) SEQ ID NOs: 3, 18, 19, 23, 25, 27, and 28; (g) SEQ ID NOs: 21, 23, 25, 27, and 28; (h) SEQ ID NOs: 3, 21, 23, 25, 27, and 28; (i) SEQ ID NO: 29; or (j) SEQ ID NOs: 3 and 29. Preferably, the third generation CAR comprises the amino acid sequence of SEQ ID NO: 29.

In an embodiment of the invention, the CAR comprises a CD8 transmembrane sequence, a 4-1BB intracellular T cell signaling sequence, and a CD3ζ intracellular T cell signaling sequence (also referred to as a “fourth generation” CAR herein). In this regard, the CAR may comprise a CD8 transmembrane amino acid sequence comprising the amino acid sequence of SEQ ID NO: 27, a 4-1BB intracellular T cell signaling amino acid sequence comprising the amino acid sequence of SEQ ID NO: 28, and a CD3ζ intracellular T cell signaling amino acid sequence comprising the amino acid sequence of SEQ ID NO: 25. In another embodiment of the invention, the fourth generation CAR may comprise the amino acid sequence(s) of (a) SEQ ID NOs: 4-9, 25, 27, and 28; (b) SEQ ID NOs: 4-9, 25, 27, 28, and 59; (c) SEQ ID NOs: 4-17, 25, 27, and 28; (d) SEQ ID NOs: 4-17, 25, 27, 28, and 59; (e) 18, 19, 25, 27, and 28; (f) SEQ ID NOs: 18, 19, 25, 27, 28, and 59; (g) SEQ ID NOs: 21, 25, 27, and 28; (h) SEQ ID NOs: 21, 25, 27, 28, and 59; (i) SEQ ID NO: 60; or (j) SEQ ID NOs: 59 and 60. Preferably, the fourth generation CAR comprises the amino acid sequence of SEQ ID NO: 60.

Included in the scope of the invention are functional portions of the inventive polypeptides, proteins, and CARs described herein. The term “functional portion,” when used in reference to a polypeptide, protein, or CAR, refers to any part or fragment of the polypeptide, protein, or CAR of the invention, which part or fragment retains the biological activity of the polypeptide, protein, or CAR of which it is a part (the parent polypeptide, protein, or CAR). Functional portions encompass, for example, those parts of a polypeptide, protein, or CAR that retain the ability to recognize target cells, or detect, treat, or prevent cancer, to a similar extent, the same extent, or to a higher extent, as the parent polypeptide, protein, or CAR. In reference to the parent polypeptide, protein, or CAR, the functional portion can comprise, for instance, about 10%, 25%, 30%, 50%, 68%, 80%, 90%, 95%, or more, of the parent polypeptide, protein, or CAR.

The functional portion can comprise additional amino acids at the amino or carboxy terminus of the portion, or at both termini, which additional amino acids are not found in the amino acid sequence of the parent polypeptide, protein, or CAR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent polypeptide, protein, or CAR.

Included in the scope of the invention are functional variants of the inventive polypeptides, proteins, or CARs described herein. The term “functional variant,” as used herein, refers to a polypeptide, protein, or CAR having substantial or significant sequence identity or similarity to a parent polypeptide, protein, or CAR, which functional variant retains the biological activity of the polypeptide, protein, or CAR of which it is a variant. Functional variants encompass, for example, those variants of the polypeptide, protein, or CAR described herein (the parent polypeptide, protein, or CAR) that retain the ability to recognize target cells to a similar extent, the same extent, or to a higher extent, as the parent polypeptide, protein, or CAR. In reference to the parent polypeptide, protein, or CAR, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the parent polypeptide, protein, or CAR.

A functional variant can, for example, comprise the amino acid sequence of the parent polypeptide, protein, or CAR with at least one conservative amino acid substitution. Alternatively or additionally, the functional variants can comprise the amino acid sequence of the parent polypeptide, protein, or CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent polypeptide, protein, or CAR.

Amino acid substitutions of the inventive polypeptides, proteins, or CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The polypeptide, protein, or CAR can consist essentially of the specified amino acid sequence or sequences described herein, such that other components, e.g., other amino acids, do not materially change the biological activity of the polypeptide, protein, CAR, functional portion, or functional variant.

The polypeptides, proteins, or CARs of embodiments of the invention (including functional portions and functional variants) can be of any length, i.e., can comprise any number of amino acids, provided that the polypeptides, proteins, or CARs (or functional portions or functional variants thereof) retain their biological activity, e.g., the ability to specifically bind to antigen, detect cancerous cells in a mammal, or treat or prevent cancer in a mammal, etc. For example, the polypeptide, protein, or CAR can be about 50 to about 5000 amino acids long, such as 50, 70, 75, 100, 125, 150, 175, 200, 300, 400, 500, 600, 700, 800, 900, 1000 or more amino acids in length.

The polypeptides, proteins, or CARs of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine ρ-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The polypeptides, proteins, or CARs of embodiments of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized.

The polypeptides, proteins, or CARs of embodiments of the invention (including functional portions and functional variants thereof) can be obtained by methods known in the art. The polypeptides, proteins, or CARs may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, e.g., Green et al., supra, and Ausubel et al., supra. Further, some of the polypeptides, proteins, or CARs of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the polypeptides, proteins, or CARs described herein (including functional portions and functional variants thereof) can be commercially synthesized by companies, such as American Peptide Company (Sunnyvale, Calif.), AmbioPharm, Inc. (North Augusta, S.C.), and Dalton Pharma Services (Toronto, Canada). In this respect, the inventive polypeptides, proteins, or CARs can be synthetic, recombinant, isolated, and/or purified.

Included in the scope of the invention are conjugates, e.g., bioconjugates, comprising any of the inventive polypeptides, proteins, CARs, anti-NY-BR-1 binding moieties, or functional portions or functional variants thereof. Conjugates, as well as methods of synthesizing conjugates in general, are known in the art (See, for instance, Hudecz, F., Methods Mol. Biol. 298: 209-223 (2005) and Kirin et al., Inorg Chem. 44(15): 5405-5415 (2005)). In this regard, an embodiment of the invention provides a conjugate comprising (a) any of the polypeptides, proteins, CARs, or anti-NY-BR-1 binding moieties described herein conjugated to (b) an effector molecule. The effector molecule may be any therapeutic molecule or a molecule that facilitates the detection of the conjugate. The effector molecule is not limited and may be any suitable effector molecule. For example, the effector molecule may be any one or more of a drug, toxin, label (e.g., any of the detectable labels described herein), small molecule, or another antibody.

Further provided by an embodiment of the invention is a nucleic acid comprising a nucleotide sequence encoding any of the polypeptides, proteins, CARs, anti-NY-BR-1 binding moieties, conjugates, or functional portions or functional variants thereof described herein. The nucleic acids of the invention may comprise a nucleotide sequence encoding any one or more of the leader sequences, linker sequences, antigen binding domains, immunoglobulin domains, transmembrane domains, and intracellular T cell signaling domains described herein. For example, the nucleic acids may comprise a nucleotide sequence encoding a leader sequence, the nucleotide sequence comprising SEQ ID NO: 30. The nucleotide sequence encoding a leader sequence may be positioned at the 5′ end of a nucleotide sequence encoding any of the polypeptides, proteins, CARs, anti-NY-BR-1 binding moieties, conjugates, or functional portions or functional variants thereof described herein. Alternatively or additionally, the nucleic acids may comprise a nucleotide sequence encoding an immunoglobulin constant domain (CH2CH3), the nucleotide sequence comprising SEQ ID NO: 57.

The nucleotide sequence may encode one or more variable regions (e.g., two variable regions) of an antigen binding domain of an anti-NY-BR-1 antibody, each variable region comprising a CDR1, a CDR2, and a CDR3. Preferably, the nucleotide sequence encoding the first variable region comprises SEQ ID NO: 31 (CDR1 of first variable region), SEQ ID NO: 32 (CDR2 of first variable region), and SEQ ID NO: 33 (CDR3 of first variable region), and the nucleotide sequence encoding the second variable region comprises SEQ ID NO: 34 (CDR1 of second variable region), SEQ ID NO: 35 (CDR2 of second variable region), and SEQ ID NO: 36 (CDR3 of second variable region). In this regard, the inventive nucleic acid can comprise the nucleotide sequences of SEQ ID NOs: 31-33, 34-36, or 31-36. Preferably, the nucleic acid comprises the nucleotide sequences of SEQ ID NOs: 31-36.

In an embodiment, the nucleic acid comprises a nucleotide sequence encoding an FR1, an FR2, an FR3, and an FR4 in addition to encoding the CDR regions described above. Preferably, the nucleotide sequence encoding the first variable region comprises SEQ ID NO: 37 (FR1 of first variable region), SEQ ID NO: 38 (FR2 of first variable region), SEQ ID NO: 39 (FR3 of first variable region), and SEQ ID NO: 40 (FR4 of first variable region), and the nucleotide sequence encoding the second variable region comprises an SEQ ID NO: 41 (FR1 of second variable region), SEQ ID NO: 42 (FR2 of second variable region), SEQ ID NO: 43 (FR3 of second variable region), and SEQ ID NO: 44 (FR4 of second variable region). In this regard, the inventive nucleic acid can comprise the nucleotide sequences of SEQ ID NOs: 37-40, 41-44, 37-44, 31-33 and 37-40, 34-36 and 41-44, or 31-44. Preferably, the nucleic acid comprises the nucleotide sequences of SEQ ID NOs: 31-44.

In an embodiment of the invention, nucleotide sequence encoding the first and second variable regions may comprise a nucleotide sequence encoding a linker amino acid sequence that joins the first and second variable regions, as described herein with respect to other aspects of the invention. The nucleotide sequence encoding the linker amino acid sequence may comprise any suitable nucleotide sequence. In an embodiment of the invention, the nucleotide sequence encoding the linker amino acid sequence may comprise the nucleotide sequence of SEQ ID NO: 45.

An embodiment of the invention provides a nucleic acid comprising a nucleotide sequence encoding any of the polypeptides, proteins, antigen binding domains, or anti-NY-BR-1 binding moieties described herein. In an embodiment, the nucleic acid comprises a nucleotide sequence encoding a scFv comprising any of the first and second variable regions joined by a linker that are described herein with respect to other aspects of the invention. In this regard, the nucleic acid may comprise the nucleotide sequence of SEQ ID NO: 46, 58 or 62.

Another embodiment of the invention provides a nucleic acid comprising a nucleotide sequence encoding any of the CARs described herein. In this regard, the nucleotide sequence encoding a CAR may comprise any of the nucleotide sequences described herein that encode any of the inventive polypeptides, proteins, antigen binding domains, or anti-NY-BR-1 binding moieties described herein. In addition, the nucleotide sequences encoding a CAR may further comprise a nucleotide sequence encoding any of the transmembrane and intracellular T cell signaling domains described herein with respect to other aspects of the invention. In an embodiment, the nucleotide sequence encoding a CAR comprises the nucleotide sequence(s) of SEQ ID NO: 47 (CD28 transmembrane and intracellular T cell signaling sequence), SEQ ID NO: 48 or 65 (CD3ζ intracellular T cell signaling sequence), SEQ ID NO: 51 or 63 (CD8 transmembrane sequence), SEQ ID NO: 52 (CD28 intracellular T cell signaling sequence), SEQ ID NO: 53 or 64 (4-1BB intracellular T cell signaling sequence), or a combination of any of the foregoing. In an embodiment of the invention, the nucleic acid comprises the nucleotide sequences of (i) SEQ ID NOs: 47-48 (second generation CAR); (ii) SEQ ID NOs: 48 and 51-53 (third generation CAR); or (iii) SEQ ID NOs: 63-65. Any of the nucleic acids described herein may further comprise, on the 5′ end, a nucleotide sequence encoding a leader sequence comprising, for example, SEQ ID NO: 30.

In an embodiment of the invention, the nucleic acid encoding a second generation CAR comprises nucleotide sequence(s) of (a) SEQ ID NOs: 31-36, 47, and 48; (b) SEQ ID NOs: 30-36, 47, and 48; (c) SEQ ID NOs: 31-44, 47, and 48; (d) SEQ ID NOs: 30-44, 47, and 48; (e) SEQ ID NOs: 30 and 49; or (f) SEQ ID NO: 49. In an embodiment of the invention, the nucleic acid encoding a third generation CAR comprises nucleotide sequence(s) of (a) SEQ ID NOs: 31-36, 48, 51-53; (b) SEQ ID NOs: 30-36, 48, and 51-53; (c) SEQ ID NOs: 31-44, 48, and 51-53; (d) SEQ ID NOs: 30-44, 48, and 51-53; (e) SEQ ID NOs: 30 and 54; or (f) SEQ ID NO: 54. In an embodiment of the invention, the nucleic acid encoding a fourth generation CAR comprises the nucleotide sequence(s) of (a) SEQ ID NOs: 31-36 and 63-65; (b) SEQ ID NOs: 31-44 and 63-65; or (c) SEQ ID NO: 61. Preferably, the nucleic acid encoding the CAR comprises the nucleotide sequence of (i) SEQ ID NO: 49; (ii) SEQ ID NO: 54; or (iii) SEQ ID NO: 61.

In some embodiments, the nucleotide sequence may be codon-optimized. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. Nucleotide SEQ ID NOs: 30-55, 57, and 61-66 described herein advantageously comprise codon-optimized sequences. In an embodiment of the invention, the nucleotide sequence is not codon-optimized. In this regard, the nucleic acid may comprise a non-codon optimized nucleotide sequence comprising SEQ ID NO: 58 (anti-NY-BR-1 scFv).

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In an embodiment of the invention, the nucleic acid may comprise complementary DNA (cDNA).

The nucleic acids of an embodiment of the invention may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can consist essentially of the specified nucleotide sequence or sequences described herein, such that other components, e.g., other nucleotides, do not materially change the biological activity of the encoded CAR, polypeptide, protein, functional portion, or functional variant.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green et al., supra. The nucleic acids can be constructed based on chemical synthesis and/or enzymatic ligation reactions using procedures known in the art. See, for example, Green et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Bio-Synthesis (Lewisville, Tex.) and Integrated DNA Technologies, Inc. (Coralville, Iowa).

The nucleic acid can comprise any isolated or purified nucleotide sequence which encodes any of the polypeptides, proteins, CARs, anti-NY-BR-1 binding moieties, conjugates, or functional portions or functional variants thereof described herein. Alternatively, the nucleotide sequence can comprise a nucleotide sequence (or combination of sequences) which is (are) degenerate to any of the sequences described herein.

An embodiment of the invention also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive polypeptides, proteins, CARs, anti-NY-BR-1 binding moieties, conjugates, or functional portions or functional variants thereof. It is generally appreciated that conditions can be rendered more stringent by the addition of increasing amounts of formamide.

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In an embodiment, the nucleic acids of the invention can be incorporated into a recombinant expression vector. In this regard, an embodiment of the invention provides recombinant expression vectors comprising any of the nucleic acids of the invention. Preferably, the recombinant expression vector comprises the nucleotide sequence of (i) SEQ ID NO: 50 (second generation CAR); (ii) SEQ ID NO: 55 (third generation CAR); or (iii) SEQ ID NO: 66 (fourth generation CAR).

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (GE Healthcare Bio-Sciences, Pittsburgh, Pa.), and the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM, and pMAMneo (Clontech). The recombinant expression vector may be a viral vector, e.g., a retroviral vector.

A number of transfection techniques are generally known in the art (see, e.g., Graham et al., Virology, 52: 456-467 (1973); Green et al., supra; Davis et al., Basic Methods in Molecular Biology, Elsevier (1986); and Chu et al., Gene, 13: 97 (1981). Transfection methods include calcium phosphate co-precipitation (see, e.g., Graham et al., supra), direct micro injection into cultured cells (see, e.g., Capecchi, Cell, 22: 479-488 (1980)), electroporation (see, e.g., Shigekawa et al., BioTechniques, 6: 742-751 (1988)), liposome mediated gene transfer (see, e.g., Mannino et al., BioTechniques, 6: 682-690 (1988)), lipid mediated transduction (see, e.g., Feigner et al., Proc. Natl. Acad. Sci. USA, 84: 7413-7417 (1987)), and nucleic acid delivery using high velocity microprojectiles (see, e.g., Klein et al., Nature, 327: 70-73 (1987)).

In an embodiment, the recombinant expression vectors of the invention can be prepared using standard recombinant DNA techniques described in, for example, Green et al., supra, and Ausubel et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may also comprise restriction sites to facilitate cloning. In some embodiments, the nucleic acid may include nucleotide sequences that include restriction sites (for example, to facilitate cloning) and which encode additional amino acid sequences that do not affect the function of the polypeptide, protein, or CAR and which may or may not be translated upon expression of the nucleic acid by a host cell (e.g., AAA).

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the polypeptides, proteins, CARs, anti-NY-BR-1 binding moieties, conjugates, or functional portions or functional variants thereof, or to the nucleotide sequence which is complementary to or which hybridizes to the nucleotide sequence encoding the inventive polypeptides, proteins, CARs, anti-NY-BR-1 binding moieties, conjugates, or functional portions or functional variants thereof. The selection of promoters, e.g., strong, weak, inducible, tissue-specific and developmental-specific, is within the ordinary skill of the artisan. Similarly, the combining of a nucleotide sequence with a promoter is also within the ordinary skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

An embodiment of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α, cell. For purposes of producing a recombinant polypeptide, protein, CAR, anti-NY-BR-1 binding moiety, conjugate, or functional portion or functional variant thereof, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a B cell or a T cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), tumor infiltrating cells, memory T cells, naïve T cells, and the like. The T cell may be a CD8⁺ T cell or a CD4⁺ T cell.

Also provided by an embodiment of the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

The polypeptides, proteins, CARs (including functional portions and variants thereof), nucleic acids, recombinant expression vectors, host cells (including populations thereof), anti-NY-BR-1 binding moieties, and conjugates, all of which are collectively referred to as “inventive anti-NY-BR-1 materials” hereinafter, can be isolated and/or purified. The term “isolated” as used herein means having been removed from its natural environment. The term “purified” or “isolated” does not require absolute purity or isolation; rather, it is intended as a relative term. Thus, for example, a purified (or isolated) host cell preparation is one in which the host cell is more pure than cells in their natural environment within the body. Such host cells may be produced, for example, by standard purification techniques. In some embodiments, a preparation of a host cell is purified such that the host cell represents at least about 50%, for example, at least about 70%, of the total cell content of the preparation. For example, the purity can be at least about 50%, can be greater than about 60%, about 70% or about 80%, or can be about 100%.

The inventive anti-NY-BR-1 materials can be formulated into a composition, such as a pharmaceutical composition. In this regard, an embodiment of the invention provides a pharmaceutical composition comprising any of the inventive anti-NY-BR-1 materials described herein and a pharmaceutically acceptable carrier. The inventive pharmaceutical compositions containing any of the inventive anti-NY-BR-1 materials can comprise more than one inventive anti-NY-BR-1 material, e.g., a CAR and a nucleic acid, or two or more different CARs. Alternatively, the pharmaceutical composition can comprise an inventive CAR material in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In a preferred embodiment, the pharmaceutical composition comprises the inventive host cell or populations thereof.

The inventive anti-NY-BR-1 materials can be provided in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which has no detrimental side effects or toxicity under the conditions of use.

The choice of carrier will be determined in part by the particular inventive CAR material, as well as by the particular method used to administer the inventive CAR material. Accordingly, there are a variety of suitable formulations of the pharmaceutical composition of the invention. Methods for preparing administrable (e.g., parenterally administrable) compositions are known or apparent to those skilled in the art and are described in more detail in, for example, Remington: The Science and Practice of Pharmacy, Pharmaceutical Press; 22nd ed. (2012).

The inventive anti-NY-BR-1 materials may be administered in any suitable manner. Preferably, the inventive anti-NY-BR-1 materials are administered by injection, (e.g., subcutaneously, intravenously, intratumorally, intraarterially, intramuscularly, intradermally, interperitoneally, or intrathecally). Preferably, the inventive anti-NY-BR-1 materials are administered intravenously. A suitable pharmaceutically acceptable carrier for the inventive anti-NY-BR-1 material for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

An “effective amount” or “an amount effective to treat” refers to a dose that is adequate to prevent or treat cancer in an individual. Amounts effective for a therapeutic or prophylactic use will depend on, for example, the stage and severity of the cancer being treated, the age, weight, and general state of health of the patient, and the judgment of the prescribing physician. The size of the dose will also be determined by the inventive anti-NY-BR-1 material selected, method of administration, timing and frequency of administration, the existence, nature, and extent of any adverse side-effects that might accompany the administration of a particular inventive anti-NY-BR-1 material, and the desired physiological effect. It will be appreciated by one of skill in the art that various cancers could require prolonged treatment involving multiple administrations, perhaps using the inventive anti-NY-BR-1 materials in each or various rounds of administration. By way of example and not intending to limit the invention, the dose of the inventive anti-NY-BR-1 material can be about 0.001 to about 1000 mg/kg body weight of the subject being treated/day, from about 0.01 to about 10 mg/kg body weight/day, about 0.01 mg to about 1 mg/kg body weight/day. When the inventive anti-NY-BR-1 material is a nucleic acid packaged in a virus, an exemplary dose of virus may be 1 ng/dose. When the inventive anti-NY-BR-1 material is a host cell or population of host cells expressing the inventive CAR, an exemplary number of inventive host cells to be administered can be about 10×10⁶ to about 10×10¹¹ cells per infusion, about 10×10⁹ cells to about 10×10¹¹ cells per infusion, or 10×10⁷ to about 10×10⁹ cells per infusion.

For purposes of the invention, the amount or dose of the inventive anti-NY-BR-1 material administered should be sufficient to effect a therapeutic or prophylactic response in the mammal over a reasonable time frame. For example, the dose of the inventive anti-NY-BR-1 material should be sufficient to bind to antigen, or detect, treat or prevent cancer in a period of from about 2 hours or longer, e.g., about 12 to about 24 or more hours, from the time of administration. In certain embodiments, the time period could be even longer. The dose will be determined by the efficacy of the particular inventive anti-NY-BR-1 material and the condition of the animal (e.g., human), as well as the body weight of the animal (e.g., human) to be treated.

For purposes of the invention, an assay, which comprises, for example, comparing the extent to which target cells are lysed and/or IFN-γ is secreted by T cells expressing the inventive anti-NY-BR-1 material upon administration of a given dose of such T cells to a mammal, among a set of mammals of which is each given a different dose of the T cells, could be used to determine a starting dose to be administered to a mammal. The extent to which target cells are lysed and/or IFN-γ is secreted upon administration of a certain dose can be assayed by methods known in the art.

When the inventive anti-NY-BR-1 materials are administered, one or more additional therapeutic agents can be coadministered to the mammal. By “coadministering” is meant administering one or more additional therapeutic agents and the inventive CAR materials sufficiently close in time such that the inventive anti-NY-BR-1 materials can enhance the effect of one or more additional therapeutic agents, or vice versa. In this regard, the inventive anti-NY-BR-1 materials can be administered first and the one or more additional therapeutic agents can be administered second, or vice versa. Alternatively, the inventive anti-NY-BR-1 materials and the one or more additional therapeutic agents can be administered simultaneously. An exemplary therapeutic agent that can be co-administered with the anti-NY-BR-1 materials is IL-2. It is believed that IL-2 enhances the therapeutic effect of the inventive anti-NY-BR-1 materials. For purposes of the inventive methods, wherein host cells or populations of cells are administered to the mammal, the cells can be cells that are allogeneic or autologous to the mammal.

It is contemplated that the inventive anti-NY-BR-1 materials and pharmaceutical compositions can be used in methods of treating or preventing cancer in a mammal. Without being bound to a particular theory or mechanism, the inventive anti-NY-BR-1 materials have biological activity, e.g., ability to recognize antigen, e.g., NY-BR-1, such that the anti-NY-BR-1 material, when expressed by a cell, is able to mediate an immune response against the cell expressing the antigen, e.g., NY-BR-1, for which the anti-NY-BR-1 material is specific. In this regard, an embodiment of the invention provides a method of treating or preventing cancer in a mammal, comprising administering to the mammal any of the polypeptides, proteins, CARs, functional portions, functional variants, nucleic acids, recombinant expression vectors, host cells, population of cells, anti-NY-BR-1 binding moieties, conjugates, and/or the pharmaceutical compositions of the invention in an amount effective to treat or prevent cancer in the mammal.

An embodiment of the invention further comprises lymphodepleting the mammal prior to administering the inventive anti-NY-BR-1 materials. Examples of lymphodepletion include, but may not be limited to, nonmyeloablative lymphodepleting chemotherapy, myeloablative lymphodepleting chemotherapy, total body irradiation, etc.

For purposes of the inventive methods, wherein host cells or populations of cells are administered, the cells can be cells that are allogeneic or autologous to the mammal. Preferably, the cells are autologous to the mammal.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the inventive methods, the cancer can be any cancer, including any of acute lymphocytic cancer, acute myeloid leukemia, rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, Ewing's sarcoma, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma), lymphoma, malignant mesothelioma, mastocytoma, melanoma, multiple myeloma, nasopharynx cancer, neuroblastoma, non-Hodgkin lymphoma, B-chronic lymphocytic leukemia, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, and ureter cancer. Preferably, the cancer is breast cancer. Preferably, the cancer is characterized by the overexpression of NY-BR-1.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment of the invention provides a use of any of the polypeptides, proteins, CARs, functional portions, functional variants, nucleic acids, recombinant expression vectors, host cells, population of cells, anti-NY-BR-1 binding moieties, conjugates, or pharmaceutical compositions of the invention for the treatment or prevention of cancer in a mammal.

Another embodiment of the invention provides a method of detecting the presence of cancer in a mammal, comprising: (a) contacting a sample comprising one or more cells from the mammal with any of the polypeptides, proteins, CARs, functional portions, functional variants, nucleic acids, recombinant expression vectors, host cells, population of cells, anti-NY-BR-1 binding moieties, or conjugates of the invention, thereby forming a complex, (b) and detecting the complex, wherein detection of the complex is indicative of the presence of cancer in the mammal.

The sample may be obtained by any suitable method, e.g., biopsy or necropsy. A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer.

With respect to an embodiment of the inventive method of detecting the presence of cancer in a mammal, the sample comprising cells of the mammal can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the mammal, e.g., the cells of any organ or tissue, including blood cells or endothelial cells.

For purposes of the inventive detecting method, the contacting can take place in vitro or in vivo with respect to the mammal. Preferably, the contacting is in vitro.

Also, detection of the complex can occur through any number of ways known in the art. For instance, the inventive CARs, polypeptides, proteins, functional portions, functional variants, nucleic acids, recombinant expression vectors, host cells, populations of cells, anti-NY-BR-1 binding moieties, or conjugates, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

Methods of testing an anti-NY-BR-1 material for the ability to recognize target cells and for antigenic specificity are known in the art. For instance, Clay et al., J. Immunol., 163: 507-513 (1999), teaches methods of measuring the release of cytokines (e.g., interferon-γ, granulocyte/monocyte colony stimulating factor (GM-CSF), tumor necrosis factor a (TNF-α) or interleukin 2 (IL-2)). In addition, anti-NY-BR-1 material function can be evaluated by measurement of cellular cytoxicity, as described in Zhao et al., J Immunol., 174: 4415-4423 (2005).

The following examples further illustrate the invention but, of course, should not be construed as in any way limiting its scope.

### Example 1

This example demonstrates NY-BR-1 mRNA expression in normal and cancerous tissues.

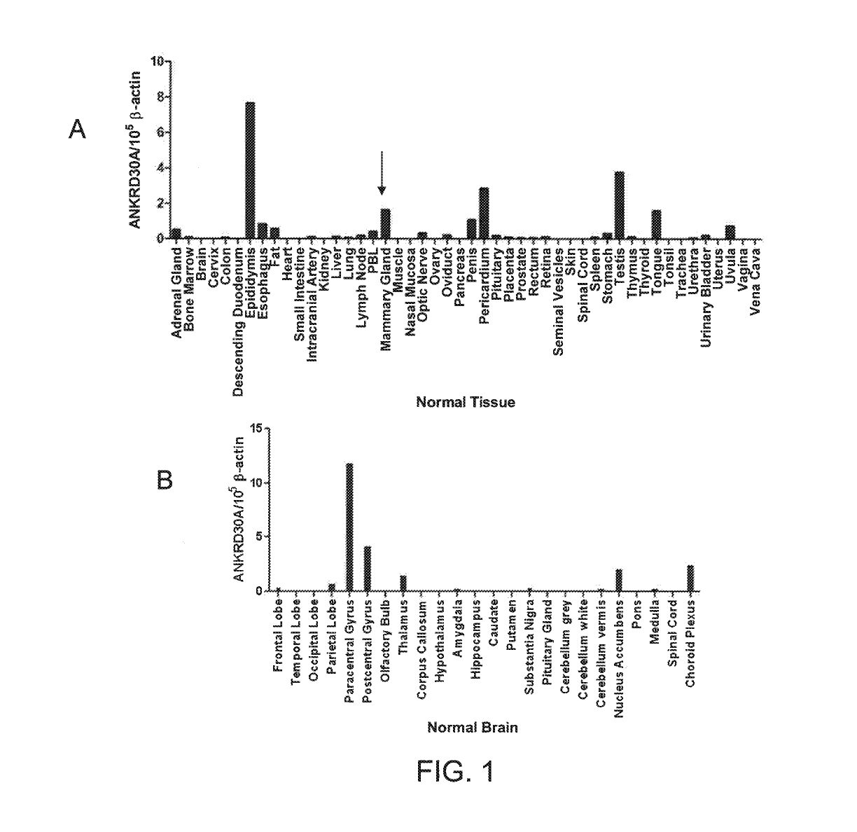

A variety of normal tissues were tested for mRNA NY-BR-1 expression using quantitative polymerase chain reaction (qPCR) and normalized to beta-actin expression. The results are shown in FIGS. 1A and 1B. The results showed that NY-BR-1 is over-expressed in normal breast tissues.

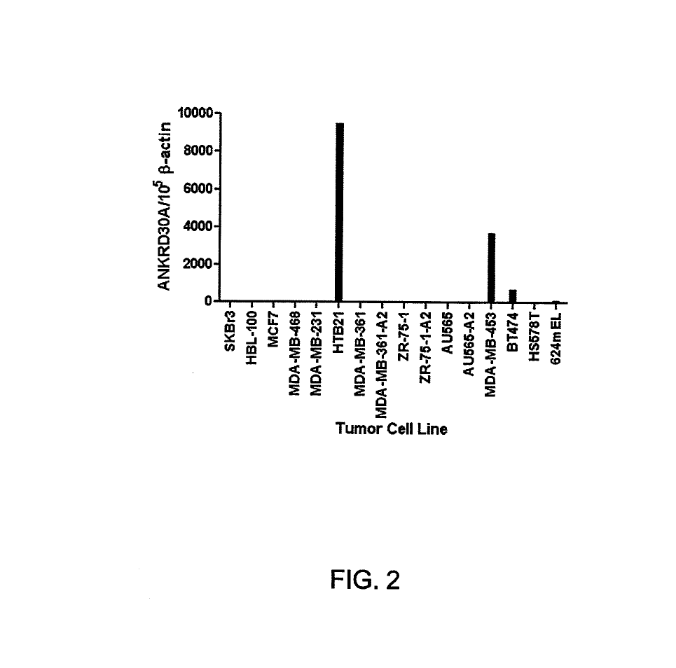

A variety of tumor cell lines were tested for NY-BR-1 mRNA expression using qPCR and reverse transcriptase (RT) PCR and normalized to beta-actin expression. The results are shown in FIG. 2. As shown in FIG. 2, HTB21 (CAMA-1), BT474, MDA-MB-453, and HTB131 (MDA-MB-453) breast cancer cell lines overexpressed NY-BR-1.

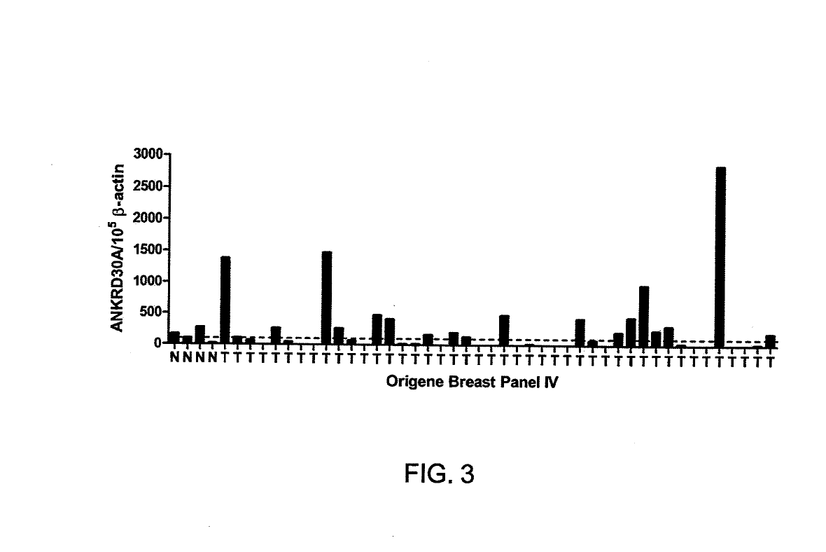

A breast cancer tissue qPCR panel (TISSUESCAN Breast Cancer Tissue qPCR Panel IV, Origene, Rockville, Md.) containing 48 tissues covering four disease stages was tested for NY-BR-1 mRNA expression using qPCR. The results are shown in FIG. 3. As shown in FIG. 3, NY-BR-1 was overexpressed in a variety of fresh breast tumor samples: 19/44 (43%) breast tumor samples and ¾ (75%) normal breast tissue samples.

### Example 2

This example demonstrates NY-BR-1 protein expression in breast cancer cell lines and NY-BR-1-transfected cells.

HTB21, HTB131, and HTB26 breast cancer cell lines were tested for NY-BR-1 protein expression using immunohistochemistry (IHC). NY-BR-1 protein expression was detected in HTB21 and HTB131, but not in the negative control (HTB26).

293T and COS cells were transduced with an expression vector encoding NY-BR-1 (293T-NY-BR-1 and COS-NY-BR-1 cells). 293T-NY-BR-1, COS-NY-BR-1, HTB21, and HTB131 cells were tested for NY-BR-1 cell surface expression using fluorescence activated cell sorting (FACS) using monoclonal antibody (mAB) NYBR1#2. Cell surface expression was detected in 293T-NY-BR-1, COS-NY-BR-1, HTB21, and HTB131 cells.

### Example 3

This example demonstrates the preparation of an anti-NY-BR-1 single chain variable fragment (scFv).

Nucleotide sequences encoding the heavy chain (SEQ ID NO: 18) and light chain (SEQ ID NO: 19) of an anti-NY-BR-1 antibody were isolated from hybridoma cell line NYBR1#2. A nucleotide sequence (SEQ ID NO: 46) encoding a scFv comprising the heavy and light chains joined by a linker amino acid sequence was prepared. A nucleotide sequence (SEQ ID NO: 30) encoding a leader sequence was included on the 5′ end of the nucleotide sequence encoding the scFv.

### Example 4

This example demonstrates the preparation of anti-NY-BR-1 CARs.

A nucleotide sequence (SEQ ID NO: 49) encoding a CAR comprising the scFv of Example 3, a CD28 transmembrane (TM) sequence, a CD28 intracellular T cell signaling sequence, and a CD3ζ intracellular T cell signaling sequence (“second generation CAR”) was prepared and cloned into an expression vector. The expression vector encoding the CAR and leader sequence comprised SEQ ID NO: 50. The CAR, including the scFv, CD28 TM sequence, CD28 signaling sequence, and CD3ζ signaling sequence, comprised an amino acid sequence comprising SEQ ID NO: 26.

A nucleotide sequence (SEQ ID NO: 54) encoding a CAR comprising the scFv of Example 3, a CD8 hinge and TM sequence, a CD28 intracellular T cell signaling sequence, a 41BB intracellular T cell signaling sequence, and a CD3ζ intracellular T cell signaling sequence (“third generation CAR”) was prepared and cloned into an expression vector. The expression vector encoding the CAR and leader sequence comprised SEQ ID NO: 55. The CAR, including the scFv, CD8 hinge and TM sequence, CD28 signaling sequence, 41BB signaling sequence, and CD3ζ signaling sequence, comprised an amino acid sequence comprising SEQ ID NO: 29.

### Example 5

This example demonstrates that cells transduced with the anti-NY-BR-1 CARs of Example 4 recognize NY-BR-1₈₅₁₋₉₂₈.

Peripheral blood lymphocytes (PBL) from two patients were transduced with an expression vector comprising SEQ ID NO: 50 (second generation CAR, or NYBR1-28Z) or SEQ ID NO: 55 (third generation CAR, or NYBR1-28BBZ). Transduction efficiency was evaluated by FACS. The results are shown in Table 1. As shown in Table 1, PBL from both patients were effectively transduced with the second and third generation CAR.

To evaluate the ability of the anti-NY-BR-1 CARs to recognize NY-BR-1₈₅₁₋₉₂₈, the CAR-expressing cells were co-cultured with 1000 or 100 ng/ml of plate-bound mesothelin (control) or immunizing peptide p77 (amino acids 851-928 of NY-BR-1). As a positive control, the CAR-expressing cells were co-cultured with the non-specific stimulator phorbol myristate acetate (PMA). As a negative control, the cells were cultured without plate-bound peptide. Interferon (IFN)-γ (pg/ml) was measured, and the results are shown in Table 1 (values underlined and in bold represent recognition of peptide). As shown in Table 1, both the second and third generation anti-NY-BR-1 CARs recognized the immunizing peptide p77 (NY-BR-1₈₅₁₋₉₂₈).

TABLE 1

| | | | | p77 (aa851-928) | | | |
|---|---|---|---|---|---|---|---|
| | | | | Plate-bound Protein (ng/ml) | | | |
| Patient PBL | CAR (HL) | % Transduction | Protein | 1000 | 100 | 0 | PMA |
| Pt. 1 | NYBR1-28BBZ | 57 | p77 | 4287 | 1889 | 8 | 6459 |
| | | | Mesothelin | 148 | 57 | | — |
| | NYBR1-28Z | 63 | p77 | 10655 | 4571 | 53 | — |
| | | | Mesothelin | 158 | 224 | | — |
| Pt. 2 | NYBR1-28BBZ | 51 | p77 | 4299 | 2114 | 83 | 8735 |
| | | | Mesothelin | 129 | 290 | | — |
| | NYBR1-28Z | 68 | p77 | 5902 | 3101 | 207 | — |
| | | | Mesothelin | 107 | 307 | | — |

### Example 6

This example demonstrates that cells transduced with the anti-NY-BR-1 CARs of Example 4 recognize NY-BR-1 transfected target cell lines.

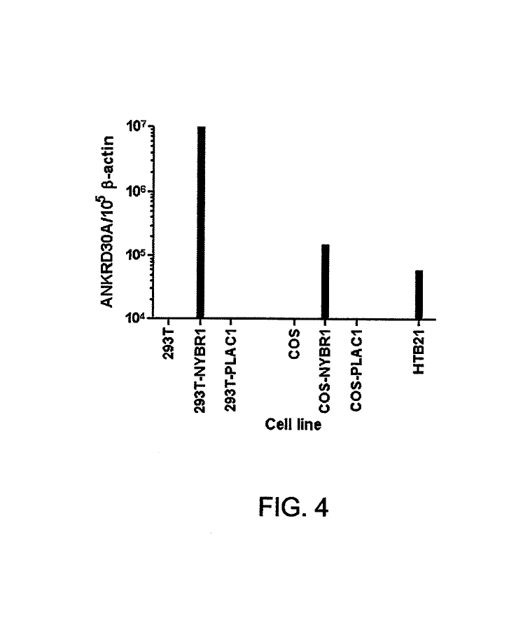

Target 293T and COS cells were transfected with a vector expressing NY-BR-1 (293T-NY-BR-1 and COS-NY-BR-1) or PLAC1 (293T-PLAC1 and COS-PLAC1). NY-BR-1 mRNA expression by the transfected cells was measured by qPCR. The results are shown in FIG. 4. As shown in FIG. 4, 293T-NYBR1, COS-NYBR1, and HTB21 cells expressed NY-BR-1 mRNA.

PBL from three patients were transiently transduced with an expression vector comprising SEQ ID NO: 50 (second generation CAR) or SEQ ID NO: 55 (third generation CAR). The ability of the CAR-expressing cells to recognize the target cell lines was evaluated upon co-culture of the CAR-expressing cells with the target cell lines. IFN-γ (pg/ml) was measured, and the results are shown in Table 2 (values underlined and in bold represent recognition of cell line). As shown in Table 2, both second and third generation anti-NY-BR-1 CARs recognize NY-BR-1-transfected cell lines but neither CAR recognized NY-BR-1-positive tumor cell line HTB21.

TABLE 2

| | | Cell Lines | | | | | | |
|---|---|---|---|---|---|---|---|---|
| | | | 293T- | 293T- | | COS- | COS- | |
| Patient | CAR (HL) | 293T | NYBR1 | PLAC1 | COS | NYBR1 | PLAC1 | HTB21 |
| Pt. 3 | NYBR1-28Z | 343 | 11403 | 1131 | 3425 | 19098 | 4747 | 1213 |
| | NYBR1-28BBZ | 776 | 5572 | 1810 | 3588 | 8786 | 4617 | 685 |
| Pt. 4 | NYBR1-28Z | 616 | 5300 | 441 | 1315 | 10992 | 2200 | 477 |
| | NYBR1-28BBZ | 409 | 3692 | 714 | 2059 | 4853 | 2199 | 400 |
| Pt. 1 | NYBR1-28Z | 979 | 9876 | 2132 | 4527 | 21840 | 5127 | 1654 |
| | NYBR1-28BBZ | 522 | 4352 | 619 | 1422 | 5752 | 1841 | 299 |

### Example 7

This example demonstrates that cells transduced with the anti-NY-BR-1 CARs of Example 4 recognize NY-BR-1 transfected target breast cancer cell lines.

Target HTB131 and HTB21 cells were transfected with a plasmid expressing NY-BR-1. The amounts of plasmid used for transfection are shown in Table 3.

PBL from three patients were transiently transduced with an expression vector comprising SEQ ID NO: 50 (second generation CAR) or SEQ ID NO: 55 (third generation CAR). The ability of the CAR-expressing cells to recognize target HTB131 or HTB21 cell lines was evaluated upon co-culture of the CAR-expressing cells with the target cell lines. IFN-γ (pg/ml) was measured, and the results are shown in Table 3 (values underlined and in bold represent recognition of cell line). As shown in Table 3, both second and third generation anti-NY-BR-1 CARs recognize NY-BR-1-transfected HTB131 and HTB21 cell lines but neither CAR recognized untransfected HTB131 or HTB21 cell lines. Without being bound to a particular theory or mechanism, it is believed that the CARs did not recognize HTB21 or HTB131 because the NY-BR-1 protein does not appear to be naturally expressed on the surface of these cells, even though they are mRNA+ for NY-BR-1, and even though IHC detected NY-BR-1 protein in HTB21 and HTB131 cells. In the IHC experiment, the cells were fixed and permeabilized, which allowed the antibody to pass through the cell membrane and stain the protein inside the cell. In the IHC experiment, the antibody may not necessarily be staining protein on the exterior of the cell. The reason that the NY-BR-1 protein does not appear to be naturally expressed on the surface of HTB21 and HTB131 cells is unknown but may relate to either some post-translational modification or splicing event.

TABLE 3

| | | [NYBR1 Plasmid] | HTB131 | | | | | HTB21 | |
|---|---|---|---|---|---|---|---|---|---|
| Patient | CAR | (ug) | 0 | 2.5 | 5 | 10 | 20 | 0 | 20 |

| | | | | | | | | |
|---|---|---|---|---|---|---|---|---|
| Pt. 3 | NYBR1-28Z | 1384 | 6198 | 6948 | 9027 | 7887 | 819 | 15784 |
| | NYBR1-28BBZ | 1591 | 2534 | 3063 | 3572 | 3989 | 1052 | 5197 |
| Pt. 4 | NYBR1-28Z | <100 | 1147 | 1231 | 1262 | 1825 | — | — |
| | NYBR1-28BBZ | <100 | 399 | 551 | 802 | 1730 | — | — |
| Pt. 1 | NYBR1-28Z | 148 | 2123 | 2404 | 2456 | 2271 | — | — |
| | NYBR1-28BBZ | 643 | 685 | 742 | 744 | 905 | — | — |

### Example 8

This example demonstrates that anti-NY-BR-1 CARs produced from packaging clones recognize NY-BR-1 target transfected cell lines.

Target cell lines were untransfected or transfected as described in Example 6.

Vector packaging cell lines were used to make a working cell bank. These cells were transiently transfected with vectors encoding the second generation or the third generation CAR of Example 4. The vector supernatant was used to transduce C5 or D7 packaging cells for the generation of a stable gammaretroviral packaging clones. The selected cell C5 and D7 clone was used to generate a master cell bank that constitutively produced retroviral vector particles.

PBL from three patients were transduced using the C5 or D7 retroviral vector particles encoding the second generation CAR or the third generation CAR of Example 4. CAR expression of the transduced PBL was confirmed by FACS.

The ability of the CAR-expressing cells to recognize targets HTB21, NY-BR-1-transfected cells, and cells that were not transfected with NY-BR-1 or PLAC1 (293T and COS) was evaluated upon co-culture of the CAR-expressing cells with the target cell lines. IFN-γ was measured, and the results are shown in Table 4 (values underlined and in bold represent recognition of cell line). As shown in Table 4, both second and third generation anti-NY-BR-1 CARs recognize NY-BR-1-transfected cell lines but neither CAR recognized NY-BR-1-positive tumor cell line HTB21.

TABLE 4

| | | Cell Lines | | | | | | |
|---|---|---|---|---|---|---|---|---|
| | | | 293T- | 293T- | | COS- | COS- | |
| Patient | CAR | 293T | NYBR1 | PLAC1 | COS | NYBR1 | PLAC1 | HTB21 |
| Pt. 3 | NYBR1-28Z (C5) | 180 | 787 | 329 | 931 | 6961 | 1157 | 197 |
| | NYBR1-28BBZ (D7) | 84 | 1204 | 170 | 592 | 2019 | 529 | 122 |
| Pt. 4 | NYBR1-28Z (C5) | 272 | 5115 | 601 | 1633 | 7225 | 2496 | 273 |
| | NYBR1-28BBZ (D7) | 163 | 1150 | 298 | 261 | 1606 | 198 | 368 |
| Pt. 1 | NYBR1-28Z (C5) | 143 | 1825 | 233 | 686 | 5431 | 871 | 494 |
| | NYBR1-28BBZ (D7) | <50 | 912 | 59 | 168 | 845 | 237 | 53 |

### Example 9

This example demonstrates that cells transduced with anti-NY-BR-1 CARs recognize NY-BR-1 transfected target cell lines.

A nucleotide sequence (SEQ ID NO: 61) encoding a CAR comprising the scFv of Example 3, a CD8 transmembrane (TM) sequence, a 4-1BB intracellular T cell signaling sequence, and a CD3ζ intracellular T cell signaling sequence (“fourth generation CAR”) was prepared and cloned into an expression vector. The expression vector encoding the CAR and leader sequence comprised SEQ ID NO: 66. The CAR, including the scFv, CD8 TM sequence, 4-1BB signaling sequence, and CD3ζ signaling sequence, comprised an amino acid sequence comprising SEQ ID NO: 60.

PBL from three patients (Patients 5-7) were transduced with an expression vector comprising SEQ ID NO: 50 (second generation CAR, or NYBR1 ScFv 28Z), SEQ ID NO: 55 (third generation CAR, or NYBR1 ScFv 28BBZ), or SEQ ID NO: 66 (fourth generation CAR, or NYBR1 ScFv BBZ). Transduction efficiency was evaluated by FACS. The results are shown in Table 5. As shown in Table 5, PBL from all three patients were effectively transduced with the second, third, or fourth generation CAR.

Cos7 cells were transfected with a plasmid encoding green fluorescent protein (GFP), NY-BR-1 (NYBR 1.1) or NY-BR-1 (NTBR1). NYBR1.1 is an isoform of NYBR1 that may be expressed in brain. Effector CAR-transduced PBL were co-cultured with target, transfected Cos7 cells. Interferon (IFN)-γ was measured. The results are shown in Table 5. As shown in Table 5, PBL transduced with the second or fourth generation CAR effectively lysed NY-BR-1 expressing target cells.

TABLE 5

| | | | IFN-γ secretion (pg/ml) upon | | |
|---|---|---|---|---|---|
| | | | co-culture with Cos7 cell line | | |
| | | Transduction | transfected with plasmid: | | |
| Pa- | | Efficiency | | NYBR | |
| tient | CAR | (%) | GFP | 1.1 | NYBR1 |
| 5 | Untransduced | not | 21 | 12 | 21 |
| | | applicable | | | |
| | | (NA) | | | |
| | NYBR1ScFv | 51 | 97 | 153 | 1109 |
| | 28Z | | | | |
| | NYBR1ScFv | 50 | 0 | 0 | 51 |
| | 28BBZ | | | | |
| | NYBR1ScFv | 59 | 621 | 527 | 1618 |
| | BBZ | | | | |
| 6 | Untransduced | NA | 0 | 6 | 15 |
| | NYBR1ScFv | 46 | 8 | 51 | 555 |
| | 28Z | | | | |
| | NYBR1ScFv | 38 | 5 | 14 | 48 |
| | 28BBZ | | | | |
| | NYBR1ScFv | 46 | 334 | 411 | 929 |
| | BBZ | | | | |
| 7 | Untransduced | NA | 28 | 36 | 59 |
| | NYBR1ScFv | 61 | 117 | 518 | 3591 |
| | 28Z | | | | |
| | NYBR1ScFv | 43 | 20 | 26 | 71 |
| | 28BBZ | | | | |
| | NYBR1ScFv | 58 | 1179 | 1261 | 3292 |
| | BBZ | | | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated polypeptide comprising the amino acid sequences of SEQ ID NOs: 4-9, optionally wherein the isolated polypeptide further comprises:
(1) the amino acid sequences of SEQ ID NOs: 10-17;
(2) a linker amino acid sequence, wherein the linker amino acid sequence optionally comprises the amino acid sequence of SEQ ID NO: 20; and/or
(3) a leader amino acid sequence, wherein the leader amino acid sequence optionally comprises the amino acid sequence of SEQ ID NO: 3.

2. (canceled)

3. The isolated polypeptide of claim 1, comprising (1) the amino acid sequences of SEQ ID NOs: 18-19 or (2) the amino acid sequence of SEQ ID NO: 21.

4. An isolated protein comprising a first polypeptide chain comprising the amino acid sequences of SEQ ID NOs: 4-6 and a second polypeptide chain comprising the amino acid sequences of SEQ ID NOs: 7-9, optionally wherein:
(1) the first polypeptide chain further comprises the amino acid sequences of SEQ ID NOs: 10-13 and the second polypeptide chain further comprises the amino acid sequences of SEQ ID NOs: 14-17;
(2) the isolated protein further comprises a linker amino acid sequence, wherein the linker amino acid sequence optionally comprises the amino acid sequence of SEQ ID NO: 20; and/or
(3) the isolated protein further comprises a leader amino acid sequence, wherein the leader amino acid sequence optionally comprises the amino acid sequence of SEQ ID NO: 3.

5. (canceled)

6. The isolated protein of claim 4, wherein the first polypeptide chain comprises the amino acid sequence of SEQ ID NO: 18 and the second polypeptide chain comprises the amino acid sequence of SEQ ID NO: 19.

7-11. (canceled)

12. A chimeric antigen receptor (CAR) comprising an antigen binding domain having antigenic specificity for NY-BR-1, a transmembrane domain, and an intracellular T cell signaling domain.

13. The CAR according to claim 12, wherein the antigen binding domain has antigenic specificity for the NY-BR-1 amino acid sequence of SEQ ID NO: 1 or SEQ ID NO: 2.

14. (canceled)

15. A CAR comprising an antigen binding domain having antigenic specificity for NY-BR-1, a transmembrane domain, and an intracellular T cell signaling domain, wherein the antigen binding domain comprises the polypeptide of claim 1.

16. The CAR according to claim 12, wherein the transmembrane domain comprises (i) a CD28 transmembrane amino acid sequence, (ii) a CD8 transmembrane amino acid sequence, or both (i) and (ii), optionally wherein the CD28 transmembrane amino acid sequence comprises the amino acid sequence of SEQ ID NO: 22 and the CD8 transmembrane amino acid sequence comprises the amino acid sequence of SEQ ID NO: 27.

17. (canceled)

18. The CAR according to claim 12, wherein the intracellular T cell signaling domain comprises a CD28 intracellular T cell signaling amino acid sequence, a CD3ζ intracellular T cell signaling amino acid sequence, a 4-1BB intracellular T cell signaling amino acid sequence, or a combination of two or more of the foregoing, optionally wherein the CD28 intracellular T cell signaling sequence comprises the amino acid sequence of SEQ ID NO: 23, the CD3ζ intracellular T cell signaling sequence comprises the amino acid sequence of SEQ ID NO: 25, and the 4-1BB intracellular T cell signaling sequence comprises the amino acid sequence of SEQ ID NO: 28.

19. (canceled)

20. The CAR according to claim 12, wherein the CAR comprises:
(i) a CD28 transmembrane amino acid sequence comprising the amino acid sequence of SEQ ID NO: 22, a CD28 intracellular T cell signaling amino acid sequence comprising the amino acid sequence of SEQ ID NO: 23, and a CD3ζ intracellular T cell signaling amino acid sequence comprising the amino acid sequence of SEQ ID NO: 25;
(ii) a CD8 transmembrane amino acid sequence comprising the amino acid sequence of SEQ ID NO: 27, a CD28 intracellular T cell signaling amino acid sequence comprising the amino acid sequence of SEQ ID NO: 23, a 4-1BB intracellular T cell signaling amino acid sequence comprising the amino acid sequence of SEQ ID NO: 28, and a CD3ζ intracellular T cell signaling amino acid sequence comprising the amino acid sequence of SEQ ID NO: 25;
(iii) a CD8 transmembrane amino acid sequence comprising the amino acid sequence of SEQ ID NO: 27, a 4-1BB intracellular T cell signaling amino acid sequence comprising the amino acid sequence of SEQ ID NO: 28, and a CD3ζ intracellular T cell signaling amino acid sequence comprising the amino acid sequence of SEQ ID NO: 25;
(iv) the amino acid sequences of: (a) SEQ ID NOs: 4-9, 22, 23, and 25; (b) SEQ ID NOs: 4-9, 24, and 25; (c) SEQ ID NOs: 3-9, 22, 23, and 25; (d) SEQ ID NOs: 3-9, 24, and 25; (e) SEQ ID NOs: 4-17, 22, 23, and 25; (f) SEQ ID NOs: 4-17, 24, and 25; (g) SEQ ID NOs: 3-17, 22, 23, and 25; (h) SEQ ID NOs: 3-17, 24, and 25; (i) SEQ ID NOs: 18, 19, 22, 23, and 25; (j) SEQ ID NOs: 18, 19, 24, and 25; (k) SEQ ID NOs: 3, 18, 19, 22, 23, and 25; (l) SEQ ID NOs: 3, 18, 19, 24, and 25; (m) SEQ ID NOs: 21, 22, 23, and 25; (n) SEQ ID NOs: 21, 24, and 25; (o) SEQ ID NOs: 3, 21, 22, 23, and 25; (p) SEQ ID NOs: 3, 21, 24, and 25; (q) SEQ ID NO: 26; or (r) SEQ ID NOs: 3 and 26;
(v) the amino acid sequences of: (a) SEQ ID NOs: 4-9, 23, 25, 27, and 28; (b) SEQ ID NOs: 3-9, 23, 25, 27, and 28; (c) SEQ ID NOs: 4-17, 23, 25, 27, and 28; (d) SEQ ID NOs: 3-17, 23, 25, 27, and 28; (e) 18, 19, 23, 25, 27, and 28; (f) SEQ ID NOs: 3, 18, 19, 23, 25, 27, and 28; (g) SEQ ID NOs: 21, 23, 25, 27, and 28; (h) SEQ ID NOs: 3, 21, 23, 25, 27, and 28; (i) SEQ ID NO: 29; or (j) SEQ ID NOs: 3 and 29;
(vi) the amino acid sequences of: (a) SEQ ID NOs: 4-9, 25, 27, and 28; (b) SEQ ID NOs: 4-9, 25, 27, 28, and 59; (c) SEQ ID NOs: 4-17, 25, 27, and 28; (d) SEQ ID NOs: 4-17, 25, 27, 28, and 59; (e) 18, 19, 25, 27, and 28; (f) SEQ ID NOs: 18, 19, 25, 27, 28, and 59; (g) SEQ ID NOs: 21, 25, 27, and 28; (h) SEQ ID NOs: 21, 25, 27, 28, and 59; (i) SEQ ID NO: 60; or (j) SEQ ID NOs: 59 and 60; or
(vii) the amino acid sequence of: (a) SEQ ID NO: 26; (b) SEQ ID NO: 29; or (c) SEQ ID NO: 60.

21.-22. (canceled)

23. An anti-NY-BR-1 binding moiety comprising the polypeptide of claim 1, wherein the anti-NY-BR-1 binding moiety is an antibody, Fab fragment (Fab), F(ab′)2 fragment, diabody, triabody, tetrabody, single-chain variable region fragment (scFv), or disulfide-stabilized variable region fragment (dsFv).

24. A conjugate comprising (a) the polypeptide of claim 1 conjugated to (b) an effector molecule, optionally wherein the effector molecule is a drug, toxin, label, small molecule, or an antibody.

25. (canceled)

26. A nucleic acid comprising a nucleotide sequence encoding the polypeptide of claim 1.

27. The nucleic acid of claim 26, comprising:
(1) the nucleotide sequences of SEQ ID NOs: 31-36, optionally wherein the nucleic acid further comprises the nucleotide sequences of SEQ ID NOs: 37-44; or
(2) the nucleotide sequence of SEQ ID NO: 46, 58, or 62.

28-33. (canceled)

34. A recombinant expression vector comprising the nucleic acid of claim 26.

35. (canceled)

36. An isolated host cell comprising the recombinant expression vector of claim 34.

37. A population of cells comprising at least one host cell of claim 36.

38. A pharmaceutical composition comprising the polypeptide of claim 1 and a pharmaceutically acceptable carrier.

39. A method of detecting the presence of cancer in a mammal, comprising:
(a) contacting a sample comprising one or more cells from the mammal with the polypeptide of claim 1, thereby forming a complex, and
(b) detecting the complex, wherein detection of the complex is indicative of the presence of cancer in the mammal.

40. (canceled)

41. A method of treating or preventing cancer in a mammal, the method comprising administering to the mammal the polypeptide according to claim 1 in an amount effective to treat or prevent cancer in the mammal.
